(19) United States
(12) Patent Application Publication
(10) Pub. No.: US 2012/0316421 A1
(43) Pub. Date: Dec. 13, 2012

(54) SYSTEM AND METHOD FOR AUTOMATED DISEASE ASSESSMENT IN CAPSULE ENDOSCOPY

(75) Inventors: Rajesh Kumar, Baltimore, MD (US); Themistocles Dassopoulos, Town and Country, MO (US); Hani Girgis, Baltimore, MD (US); Gregory Hager, Baltimore, MD (US)

(73) Assignee: The Johns Hopkins University, Baltimore, MD (US)

(21) Appl. No.: 13/382,855

(22) PCT Filed: Jul. 7, 2010
(86) PCT No.: PCT/US10/41220

§ 371 Date: Jun. 20, 2012

Related U.S. Application Data
(60) Provisional application No. 61/223,585, filed on Jul. 7, 2009.

(51) Int. Cl. A61B 6/00; H04N 7/18

(52) U.S. Cl. 600/407; 348/65

(57) ABSTRACT

A system and method for automated image analysis which may enhance, for example, capsule endoscopy diagnosis. The system and methods may reduce the time required for diagnosis, and also help improve diagnostic consistency using an interactive feedback tool. Furthermore, the system and methods may be applicable to any procedure where efficient and accurate visual assessment of a large set of images is required.

### CROSS-REFERENCE TO RELATED APPLICATION

This application claims priority to U.S. Provisional Application No. 61/223,585 filed Jul. 7, 2009, the entire content of which is hereby incorporated by reference.

### FEDERAL FUNDING

This invention was made with U.S. Government support of Grant No. 5R21EB008227-02, awarded by National Institutes of Health. The U.S. Government has certain rights in this invention.

### BACKGROUND

1. Field of Invention

The current invention relates to systems and methods of processing images from an endoscope, and more particularly automated systems and methods of processing images from an endoscope.

2. Discussion of Related Art

The contents of all references, including articles, published patent applications and patents referred to anywhere in this specification are hereby incorporated by reference.

There have been several capsules developed for “blind” collection of diagnostic data in the GI tract. For example the Medtronic Bravo (recently acquired by GIVEN) has been developed to make simple chemical measurements (e.g. pH). The clinical utility of these capsules has been limited due to the lack of accurate anatomical localization and visualization. More recent wireless Capsule Endoscopy (CE) allows visual imaging access into the gastrointestinal (GI) tract, especially the small bowel. A disposable CE capsule system, for example, consists of a small color camera, lighting electronics, wireless transmitter, and a battery. The first small bowel capsule (the PillCam small bowel (SB) M2A, GIVEN Imaging Inc.) measured 26 mm in length and 11 mm in diameter. Similarly sized competing capsules (e.g. the clinically approved Olympus EndoCapsule) have since been introduced. Prototype capsules still under development include new features such as active propulsion and wireless power transmission, and are designed for imaging the small bowel, the stomach, and the colon.

Wireless Capsule Endoscopy (CE) allows visual imaging access into the gastrointestinal (GI) tract. A CE system FIG. 1, 110 and 120 (G. Iddan, G. Meron, A. Glukhovsky, and P. Swain, “Wireless capsule endoscopy,” Nature, vol. 405, no. 6785, pp. 417, 2000) includes a small color camera, light source, wireless transmitter, and a battery in a capsule only slightly larger than a common vitamin pill. The capsule is taken orally, and is propelled by peristalsis along the small intestine. It transmits approximately 50,000 images over the course of 8 hours, using radio frequency communication. The images may be stored on an archiving device, consisting of multiple antennae and a portable storage system, attached to the patient's abdomen for the duration of the study. Upon completion, the patient may return the collecting device to the physician who transfers the accumulated data to the reviewing software on a workstation for assessment and interpretation. Due to limitations in the power supply of the capsule, image resolution (576×576) as well as the video frame rate (2 fps) are low. This makes evaluation of data a tedious and time consuming (usually 1-2 hours) process. Clinicians typically require more than one view of a pathology for evaluation. The current software (Given Imaging, “Given imaging ltd.,” http://www.givenimaging.com, March 200) may allow for consecutive frames to be viewed simultaneously. However, due to the low frame rate, neighboring images may not necessarily contain the same areas of interest and the clinician is typically left toggling between images in the sequence, thus making the process even more time consuming.

Unlike endoscopy, CE is a non-invasive outpatient procedure. Upon completion of an examination, the patient returns the collecting device to the physician who transfers the accumulated data to the reviewing software on a workstation for assessment and interpretation.

The capsule analysis software from the manufacturers includes features for detecting luminal blood, image structure enhancement, simultaneous multiple sequential image views, and variable rate of play-back of the collected data. Blood and organ boundary detection have been a particular focus of interest.

The typical CE study reading time is reported to be one to two hours. In addition to being a tedious and time consuming process, detection rates may also vary among clinicians, especially for early stage pathology. Features for reducing assessment time, including variable rate video playback and multiple simultaneous image frame views (1-4), have been investigated both by capsule manufacturers and in the literature. However, these have proven to be of limited benefit.

As CE grows in popularity and as miniaturized sensors and imagers improve, there will be a commensurate growth in the amount of CE data that must be evaluated. There is thus a corresponding need to improve the effectiveness, efficiency, and quality of CE diagnosis by reducing reading time and complexity, and by improving accuracy and consistency of assessment of CE studies. There is a clear role and need for computational support methods, including machine learning and computer vision, to improve off-line analysis and facilitate more accurate and consistent diagnosis.

### SUMMARY

An automated method of processing images from an endoscope according to an embodiment of the current invention includes receiving one or more endoscopic images by an image processing system, processing each of the endoscopic images with the image processing system to determine whether at least one attribute of interest is present in each image that satisfies a predetermined criterion, and classifying the endoscopic images into a reduced set of images each of which contains at least one attribute of interest and a remainder set of images each of which is free from the attribute.

An endoscopy system according to an embodiment of the current invention includes an endoscope and a processing unit in communication with the endoscope. The processing unit includes executable instructions for detecting an attribute of interest. In response to receiving a plurality of endoscopic images from the endoscope and based on the executable instructions, the processing unit performs a determination of whether at least one attribute of interest is present in each image that satisfies a predetermined criterion and the processing unit performs a classification of the plurality of endoscopic images into a reduced set of images each of which contains at least one attribute of interest and a remainder set of images each of which is free from at least one attribute of interest.

In yet another embodiment of the current invention, a computer readable medium stores executable instructions for execution by a computer having memory. The medium stores instructions for receiving one or more endoscopic images, processing each of the endoscopic images to determine whether at least one attribute of interest is present in each image that satisfies a predetermined criterion, and classifying the endoscopic images into a reduced set of images each of which contains at least one attribute of interest and a remainder set of images each of which is free from at least one attribute of interest.

### BRIEF DESCRIPTION OF THE DRAWINGS

The invention may be better understood by reading the following detailed description with reference to the accompanying figures, in which:

FIG. 1 depicts conventional endoscopy imaging devices;

FIG. 2 depicts illustrative images from endoscopy imaging devices;

FIG. 3 depicts illustrative images from endoscopy imaging devices showing Crohn's disease lesions of increasing severity;

FIG. 4 depicts illustrative images from endoscopy imaging devices;

FIG. 5 depicts illustrative images from endoscopy imaging devices with a region of interest highlighted;

FIG. 6 depicts an illustrative CE image represented by 6 DCD prominent colors, and an edge intensity image with 2×2 sub-blocks for EHD filters;

FIG. 7 depicts an illustrative graph showing Boosted Registration Results;

FIG. 8 depicts an example of information flow in an embodiment of the current invention;

FIG. 9 depicts illustrative images from endoscopy imaging devices showing the same lesion in different images and a ranking of lesion severity;

FIG. 10 depicts illustrative images from endoscopy imaging devices where the images are ranked in increasing severity;

FIG. 11 depicts illustrative images from endoscopy imaging devices where the images are ranked in increasing severity;

FIG. 12 depicts an expanded view of feature extraction according to an embodiment of the current invention;

FIG. 13 depicts illustrative lesion images and the effect of using adaptive thresholds on the edge detectors responses;

FIG. 14 depicts an illustrative information flow diagram that may be used in implementing an embodiment of the present invention;

FIG. 15 depicts an example of a computer system that may be used in implementing an embodiment of the present invention;

FIG. 16 depicts an illustrative imaging capture and image processing and/or archiving system according to an embodiment of the current invention;

FIG. 17 depicts an illustrative metamatching procedure that may be used in implementing an embodiment of the current invention;

FIG. 18 depicts an illustrative screen shot of a user interface application that may be used in implementing an embodiment of the present invention;

FIG. 19 depicts a sample graph showing estimated ranks vs. feature vector sum (Σ, ƒ) for simulated data;

FIG. 20 depicts disc images sorted (left to right) by estimated ranks;

FIG. 21 depicts illustrative endometrial images;

FIG. 22 depicts a table showing sample SVM accuracy rates; and

FIG. 23 depicts a table showing sample SVM recall rates.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention.

All references cited herein are incorporated by reference as if each had been individually incorporated.

In one embodiment of the invention an automated method of processing images from an endoscope is disclosed. The method may include receiving endoscopic images and processing each of the endoscopic images to determine whether an attribute of interest is present in each image that satisfies a predetermined criterion. The method may also classify the endoscopic images into a set of images that contain at least one attribute of interest and a remainder set of images which do not contain an attribute of interest.

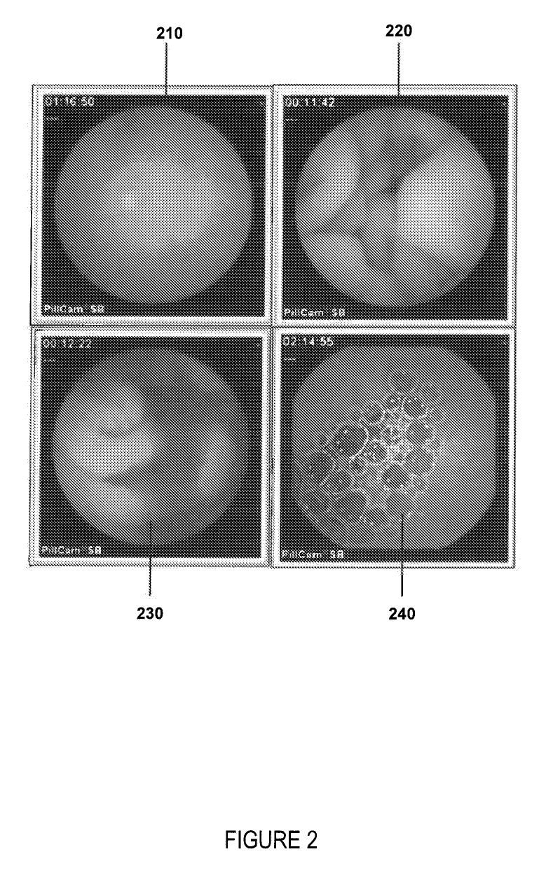

FIG. 2 depicts some sample images of the GI tract using CE. In FIG. 2, 210 depicts a Crohn's lesion, 220 depicts normal villi, 230 shows bleeding obscuring details of the GI system, and 240 shows air bubbles.

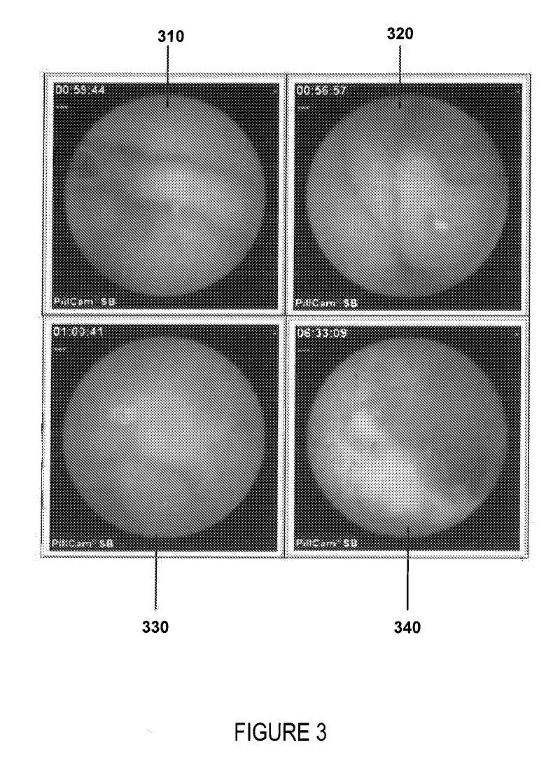

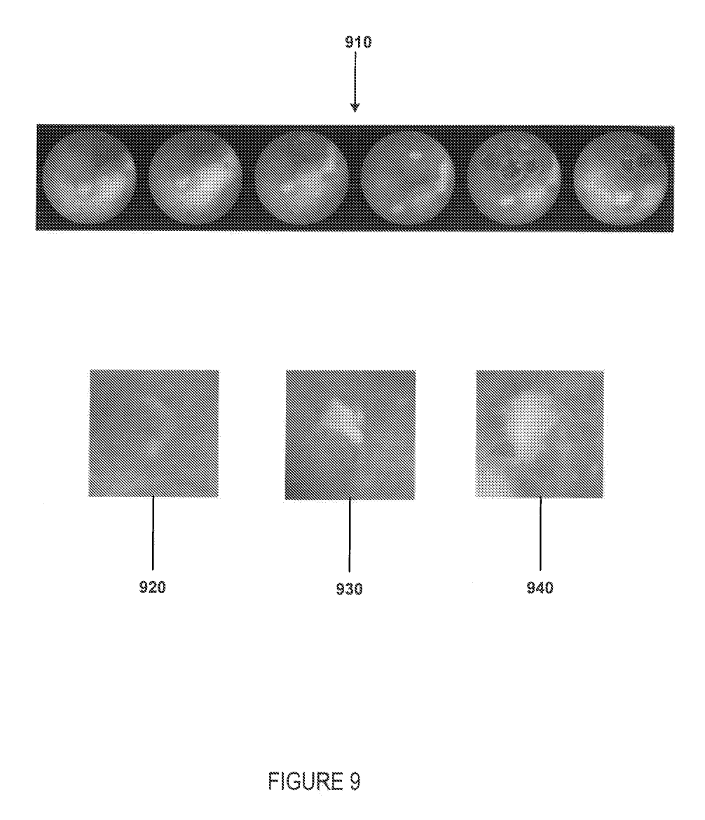

Crohn's disease (CD) is an inflammatory bowel disease (IBD) that develops when individuals with a genetic predisposition are exposed to environmental triggers. Currently, the environmental triggers are poorly defined. CD can affect any part of the gastrointestinal tract (upper GI tract, small bowel and/or colon), although it more frequently affects the ileum and/or the colon. The mucosal inflammation is characterized by discrete, well-circumscribed (“punched-out”) erosions and ulcers. More severe mucosal disease progresses to submucosal inflammation, leading to complications, such as strictures, fistulae and perforation. In FIG. 3, 310, 320, 330, and 340 depict images of CD lesions of increasing severity as also shown in FIG. 9, 920, 930, and 940.

The quality of CE images may be highly variable due to its peristalsis propulsion, complexity of GI structures and contents of the GI tract, as well as limitations of the disposable imager itself 110, 120. As a result, only a relatively small percentage of images actually contribute to the clinical diagnosis. Recent research has focused on developing methods for reducing the complexity and time needed for CE diagnosis by removing unusable images or detecting images of interest. Recent methods of using color information and applying it on data from 3 CE studies to isolate “non-interesting” images containing excessive food or fecal matter or air bubbles (Md. K. Bashar, K. Mori, Y, Suenaga, T. Kitasaka, Y. Mekada, “Detecting Informative Frames from Wireless Capsule Endoscopic Video Using Color and Texture Features”, in Proc MICCAI, Springer Lecture Notes In Computer Science (LNCS), vol. 5242, pp. 603-611, 2008). These methods have been compared with Gabor and discrete wavelet feature methods. Others describe a method for analyzing motion detected between the frames using principal component analysis to create higher order motion data (L. Igual, S. Segui, J. Vitria, F. Azpiroz, and P. Radeva, “Eigenmotion-Based Detection of Intestinal Contractions”, in Proc. CAIP, Springer LNCS, vol. 4673, pp. 293-300, 2007). They then use relevance vector machine (RVM) methods to classify contraction sequences.

Some have applied expectation maximization (EM) clustering on a dataset of around 15,000 CE images for blood detection (S. Hwang, J. Oh, J. Cox, S. J. Tang, H. F. Tibbals. “Blood detection in wireless capsule endoscopy using expectation maximization clustering”, in Proc. SPIE, Vol. 6144. 2006). A blood detection method has been reported (Y. S. Jung, Y. H. Kim, D. H. Lee, J. H. Kim, “Active Blood Detection in a High Resolution Capsule Endoscopy using Color Spectrum Transformation” in Proc. International Conference on BioMedical Engineering and Informmatics, pp. 859-862, 2008). The capsule analysis software from a manufacturer also includes a feature for detecting luminal blood. Also presented is a method for detecting GI organ boundaries (esophagus, stomach, duodenum, jejunum, ileum and colon) using energy functions (J. Lee, J. Oh, S. K. Shah, X. Yuan, S. J. Tang, “Automatic Classification of Digestive Organs in Wireless Capsule Endoscopy Videos”, in Proc. SAC'07, 2007). In addition, other groups have investigated improving CE diagnosis (M. Coimbra, P. Campos, J. P. Silva Cunha; “Topographic segmentation and transit time estimation for endoscopic capsule exams”, in Proc. IEEE ICASSP, 2006; D. K. Iakovidisa, D. E. Maroulisa, S. A, Karkanis; “An intelligent system for automatic detection of gastrointestinal adenomas in video endoscopy”, Computers in biology and medicine; M. M. Zheng, S. M. Krishnan, M. P. Tjoa; “A fusion-based clinical decision support for disease diagnosis from endoscopic images”, Computers in biology and medicine, vol. 35 pp. 259-274, 2005; J. Berens, M. Mackiewicz, D. Bell. “Stomach, intestine and colon tissue discriminators for wireless capsule endoscopy images”, in Proc. SPIE Confrrence on Medical Imaging, vol. 5747, pp. 283-290, 2005; H. Vu, T. Echigo, R. Sagawa. K. Yagi, M. Shiba, K. Hiiguchi, T. Arakawa, Y. Yagi “Contraction Detection in Small Bowel from an Image Sequence of Wireless Capsule Endoscopy”, in Proc. MICCAI, LNCS, vol. 4791, pp. 775-783, 2007).

Methods for statistical classification, including motion data into surgical gestures using LDA, Support Vector Machines, and Hidden Markov models, and applying these and other statistical learning algorithms to a variety of computer vision problems may be helpful (Lin, H. C., I. Shafran, T. Murphy, A. M. Okamura, D. D. Yuh, G. D. Hager: “Automatic Detection and Segmentation of Robot-Assisted Surgical Motions” in Proc. MICCAI, LNCS, vol. XYZW, pp. 802-810, 2005; L. Lu, G. D. Hager, L. Younes, “A Three Tiered Approach for Articulated Object Action Modeling and Recognition”, Advances in Neural Information Processing Systems, vol. 17, pp. 841-848, 2005. L. Lu, K. Toyama, G. D. Hager, “A Two Level Approach for Scene Recognition”, in Proc. IEEE Conference on Computer Vision and Pattern Recognition, pp. 688-695, 2005).

One embodiment of the invention includes a tool for semi-automated, quantitative assessment of pathologic findings, such as, for example, lesions that appear in Crohn's disease of the small bowel. Crohn's disease may be characterized by discrete, identifiable and well-circumscribed (“punched-out”) erosions and ulcers. More severe mucosal disease predicts a more aggressive clinical course and, conversely, mucosal healing induced by anti-inflammatory therapies is associated with improved patient outcomes. Automated analysis may begin with the detection of abnormal tissue.

In one embodiment of the invention, automated detection of lesions and classification are performed using machine learning algorithms, Traditional classification and regression techniques may be utilized as well as rank learning or Ordinal regression. The application of machine learning algorithms to image data may involve the following steps: (1) feature extraction, (2) dimensionality reduction, (3) training, and (4) validation.

### Feature Extraction

One embodiment of this invention includes (1) represent the data in a format where inherent structure is more apparent (for the learning task), (2) reduce the dimensions of the data, and (3) create a uniform feature vector size for the data (i.e., for example, images of different sizes will still have a feature vector of the same size). Images exported from CE for automated analysis may suffer from compression artifacts, in addition to noise resulting from the wireless transmission. Methods used for noise reduction include linear and nonlinear filtering and dynamic range adjustments such as histogram equalization (M. Sonka, V. Hlavac, and R. Boyle. Image Processing, Analysis, and Machine Vision. Thomson-Engineering, 2007).

One embodiment of this invention include wide range of color, edge, texture and visual features, such as those used in the literature for creation of higher level representations of CE images as described in the following. Coimbra et al. use MPEG-7 visual descriptors as feature vectors for their topographic segmentation system (M. Coimbra, P. Campos, and J. P. S. Cunha. Topographic segmentation and transit time estimation for endoscopic capsule exams. In Proceedings of IEEE International Conference on Acoustics, Speech and Signal Processing, volume 2, pages II-II, May 2006; BS Manjunath, JR Ohm, VV Vasudevan, and A Yamada. Color and texture descriptors. IEEE Transactions on circuits and systems for video technology, 11(6):703-715, 2001). Lee et al. utilize hue, saturation and intensity (HSI) color features in their topographic segmentation system (J. Lee, J. Oh, S. K. Shah, X. Yuan, and S. J. Tang. Automatic classification of digestive organs in wireless capsule endoscopy videos. In SAC '07: Proceedings of the 2007 ACM symposium on Applied computing, pages 1041-1045, New York, N.Y., USA, 2007. ACM). Vu et al. use edge features for contraction detection (H. Vu, T. Echigo, R. Sagawa, K. Yagi, M. Shiba, K. Higuchi, T. Arakawa, and Y. Yagi. Contraction detection in small bowel from an image sequence of wireless capsule endoscopy. In Proceedings of MICCAI, Lecture Notes in Computer Science, volume 4791, pages 775-783, 2007). Color and texture features are used by Zheng et al. in their decision support system (M. M. Zheng, S. M. Krishnan, and M. P. Tjoa. A fusion-based clinical decision support for disease diagnosis from endoscopic images. Computers in Biology and Medicine, 35(3):259-274, 2005). Color histograms are also utilized along with MPEG-7 visual descriptors, Haralick texture features, and a range of other features (S. Bejakovic, R. Kumar, T. Dassopoulos, G. Mullin, and G. Hager. Analysis of crohns disease lesions in capsule endoscopy images. In International Conference on Robotics and Automation, ICRA, pages 2793-2798, May 2009; R. Kumar, P. Rajan, S. Bejakovic, S. Seshamani, G. Mullin, T. Dassopoulos, and G. Hager. Learning disease severity for capsule endoscopy images. In IEEE ISBI 2009, accepted, 2009; S. Seshamani, P. Rajan, R. Kumar, H. Girgis, G. Mullin, T. Dassopoulos, and G. D. Hager. A boosted registration framework for lesion matching. In Medical Image Computing and Computer Assisted Intervention (MICCAI), accepted, 2009; S. Seshamani, R. Kumar, P. Rajan, S. Bejakovic, G. Mullin, T. Dassopoulos, and G. Hager. Detecting registration failure. In IEEE ISBI 2009, accepted, 2009).

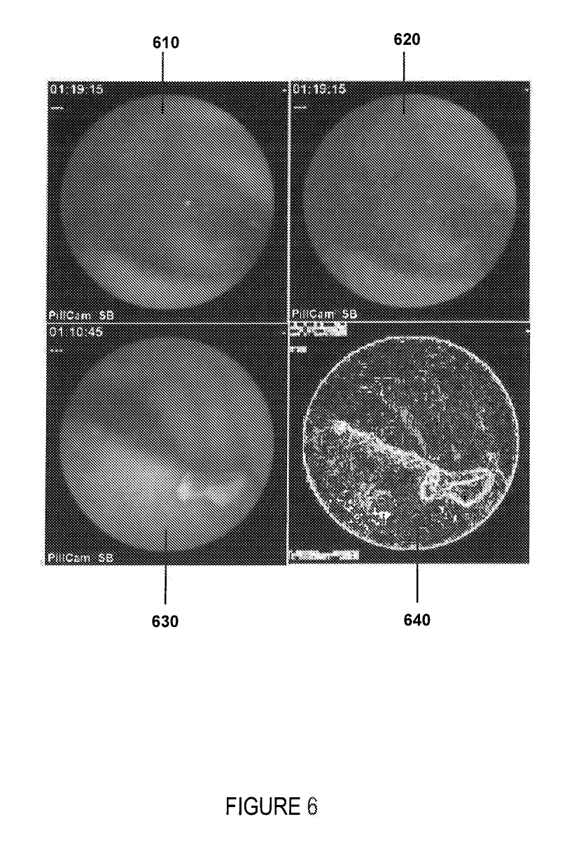

In one embodiment of the invention, a Dominant Color Descriptor (DCD) is used which clusters neighboring colors into a small number of clusters. This DCD feature vector may include the dominant colors, and their variances, and for edges the Edge Histogram Descriptor (EHD) may be used which uses 16 non-overlapping bins, for example, accumulating edges in the 0±, 45±, 90±, 135± directions and non-directional edges for a total of 80 bins. FIG. 6 shows images 610 and 630 and their DCD 620 and EHD 640 reconstructions. In an embodiment MPEG-7 Homogeneous Texture Descriptor (HTD), and Haralick statistics may be used. HTD may use a bank of Gabor filters containing 30 filters, for example, which may divide the frequency space into 30 channels (6 sections in the angular direction×5 sections in the radial direction), for example. Haralick statistics may include measures of energy, entropy, maximum probability, contrast, inverse difference moment, correlation, and other statistics. Also color histograms (RGB, HSI, and Intensity), and other image measures extracted from CE images as feature vectors may be used.

### Dimensionality Reduction

One embodiment of the invention includes dimensionality reduction. When several types of feature vectors are combined, feature data is still usually high-dimensional and may contain several redundancies. Dimensionality reduction may involve the conversion of the data into a more compact representation. Dimensional reduction may allow the visualization of data, greatly aiding in understanding the problem under consideration. For example, through data visualization one can determine the number of clusters in the data or if the classes are linearly or non-linearly separable. Also, the elimination of redundancies and reduction in size of the data vector may greatly reduce the complexity of the learning algorithm applied to the data. Examples of reduction methods used in an embodiment of the invention include, but are not limited to, Kohonen Self Organizing Maps, Principal Component Analysis, Locally Linear Embedding, and Isomap (T. Kohonen, Self-organization and associative memory: 3rd edition. Springer-Verlag New York, Inc., New York, N.Y., USA, 1989; H. Schneiderman and T. Kanade. Probabilistic modeling of local appearance and spatial relationships for object recognition. In Computer Vision and Pattern Recognition, 1998. Proceedings of the IEEE Computer Society Conference on, pages 45-51, July 1998; Matthew Turk and Alex Pentland. Eigenfaces for recognition, Journal of Cognitive Neuroscience, 3(1):71-86, 1991; Sam T. Roweis and Lawrence K. Saul. Nonlinear Dimensionality Reduction by Locally Linear Embedding, Science, 290(5500):2323-2326, 2000; Joshua B. Tenenbaum, Vin de Silva, and John C. Langford. A Global Geometric Framework for Nonlinear Dimensionality Reduction. Science, 290(5500): 2319-2323, 2000)

### Training

One embodiment of the invention includes machine learning or training including the following. There may be two main paradigms in machine learning: supervised learning and unsupervised learning. In supervised learning, each point in the data set may be associated with a label while training. In unsupervised learning, labels are not available while training but other statistical priors such as the number of expected classes may be assumed. Supervised statistical learning algorithms include Artificial Neural Networks (ANN), Support Vector Machines (SVM), and Linear Discriminant Analysis (LDA) (Christopher M. Bishop. Pattern Recognition and Machine Learning (Information Science and Statistics) Springer, August 2006; M. T. Coimbra and J. P. S. Cunha. Mpeg-7 visual descriptors contributions for automated feature extraction in capsule endoscopy. IEEE Transactions on Circuits and Systems for Video Technology, 16(5):628-637, May 2006; F Vilarino, P Spyridonos, O Pujol, J Vitria, and P Radeva. Automatic detection of intestinal juices in wireless capsule video endoscopy. In ICPR '06: Proceedings of the 18th International Conference on Pattern Recognition, pages 719-722, Washington, D.C., USA, 2006. IEEE Computer Society). For unsupervised learning, common methods may include algorithms such as the k-means and the EM (David A. Forsyth and Jean Ponce. Computer Vision: A Modern Approach. Prentice Hall, August 2002; J. A. Lasserre, C. M. Bishop, and T. P. Minka. Principled hybrids of generative and discriminative models In Computer Vision and Pattern Recognition, 2006 IEEE Computer Society Conference on, volume 1, pages 87-94, June 2006; Zhuowen Tu. Probabilistic boosting-tree: learning discriminative models for classification, recognition, and clustering. In Computer Vision, 2005. ICCV 2005. Tenth IEEE International Conference on, volume 2, pages 1589-1596, October 2005). One can apply supervised learning algorithms to solve classification and regression problems. Data clustering may be a classic unsupervised learning problem. Two powerful methods for improving classifier performance include boosting and bagging (Christopher M. Bishop. Pattern Recognition and Machine Learning (Information Science and Statistics). Springer, August 2006). Both may be methods of using several classifiers together to “vote” for a final decision. Combination rules include voting, decision trees, and linear and nonlinear combinations of classifier outputs. These approaches also provide the ability to control the tradeoff between precision and accuracy through changes in weights or thresholds. These methods naturally lend themselves to extension to large numbers of localized features.

### Validation

One embodiment of the invention includes validation of the automated system as described in the following paragraph. During training, the accuracy of the learner may be measured by the training error. However, a small training error does not guarantee a small error on unseen data. An over-fitting problem during training may occur when the chosen model may be more complex than needed, and may result in data memorization and poor generalization. A learning algorithm should be validated on an unseen portion of the data. A learning algorithm that generalizes well may have testing error similar to the training error. When the amount of labeled data is large, the data may be partitioned into three sets. The algorithm may be trained on one partition and validated on another partition. The algorithm parameters may be adjusted during training and validation. The training and the validation steps may be repeated until the learner performs well on both of the training and the validation sets. The algorithm may also be tested on the third partition (Christopher M. Bishop. Pattern Recognition and Machine Learning (Information Science and Statistics) Springer, August 2006). With limited labeled data, as is often the case in medical imaging, K-fold cross-validation method is often employed (Richard O. Duda, Peter E. Hart, and David G. Stork. Pattern Classification (2nd Edition). Wiley-Interscience, 2000). The K-fold method may divide the labeled dataset into K random partitions of about the same size, and trains the learner on K−1 of those portions, Validation may be performed on the remaining partition and the entire process may be repeated while leaving out a different partition each time. Typical values of K are on the order of 10. When K is equal to the number of data points, the validation may be referred to as the leave-one-out technique. The final system may be trained on the entire dataset. Although the exact accuracy of that system cannot be computed, it is expected to be close to, and more accurate than the system tested by the K-fold cross validation.

In one embodiment of the invention, support vector machines (SVM) are used to classify CE images into those containing lesions, normal tissue, and food, bile, stool, air bubbles, etc. (extraneous matter) (S. Bejakovic, R. Kumar, T. Dassopoulos, G. Mullin, and G. Hager. Analysis of crohns disease lesions in capsule endoscopy images. In International Conference on Robotics and Automation, ICRA, pages 2793-2798, May 2009). DCD and variances, Haralick features, EHD, and HTD feature vectors may be in one embodiment of the invention and used directly as feature vectors for binary classification (e.g., for example, lesion/nonlesion).

In one embodiment of the invention, given a region of interest (ROI), the system determines whether or not a match is found by automatic registration to another frame is truly another instance of the selected ROI. The embodiment may use the following. Using a general discriminative learning model, an ROI pair may be associated with a set of metrics (e.g., but not limited to, pixel, patch, and histogram based statistics) and train a classifier that may discriminate misregistrations from correct registrations using, for example, adaboost (R. E. Schapire and Y. Singer. Improved boosting algorithms using confidence-rated predictions. In Computational learning theory, pages 80-91, 1998; Richard O. Duda, Peter E. Hart, and David G. Stork. Pattern Classification (2nd Edition). Wiley-Interscience, 2000). The classifier may be extended with Haralick features and MPEG-7 descriptors discussed above to create a meta registration technique to boost the retrieval rate (S. Seshamani, P. Rajan, R. Kumar, H. Girgis, G. Mullin, T. Dassopoulos, and G. D. Hager. A boosted registration framework for lesion matching. In Medical Image Computing and Computer Assisted Intervention (MICCAI), accepted, 2009). After region matching using, for example, five different standard global registration methods (e.g., but not limited to, template matching, mutual information, two weighted histogram methods, and SIFT), the trained classifier may be applied to determine if any of the matches are correct. The correct matches are then ranked using ordinal regression to determine the best match. Experiments have shown that the meta-matching method outperforms any single matching method.

In one embodiment of the invention a severity assessment is accomplished through the following. A semi-automatic framework to assess the severity of Crohn's lesions may be used (R. Kumar, P. Rajan, S. Bejakovic, S. Seshamani, G. Mullin, T, Dassopoulos, and G. Hager. Learning disease severity for capsule endoscopy images. In IEEE ISBI 2009, accepted, 2009) The severity rank may be based on pairwise comparisons among representative images. Classification and ranking, have been formulated as problems of learning a map from a set of features to a discrete set of label, for example, for face detection [3], object recognition [4], and scene classification (B. S. Lewis. Expanding role of capsule endoscopy in inflammatory bowel disease. World Journal of Gastroenterology, 14(26):4137-4141, 2008; R Eliakim, D Fischer, and A Suissa. Wireless capsule endoscopy is a superior diagnostic tool in comparison to barium follow through and computerized tomography in patients with suspected crohn's disease, European J Gastroenterol Hepatol, 15:363-367, 2003; I Chermesh and R Eliakim. Capsule endoscopy in crohn's disease—indications and reservations 2008 Journal of Crohn's and Colitis, 2:107-113, 2008). In one embodiment ranking may be treated as a regression problem to find a ranking function between a set of input features and a continuous range of ranks or ssessment. Assuming a known relationship [symbol] (e.g. global severity rating mild<moderate<severe) on a set of Images I, a real-valued ranking function R may be computed such that Iₓ [symbol] I_{y}∈P [symbol] R(Iₓ)<R(I_{y}). The ranking function may be based on empirical statistics of the training set. A preference pair [symbol] x, y [symbol] ∈ P̅, where P̅ is the transitive closure of P, may be thought of as a pair of training examples for a binary classifier. For example, given,

$$B ( p ) = \{ \begin{matrix}0 & p ∈ \overline{P} \\ 1 & otherwise\end{matrix}$$

A classifier C may be trained such that for any p∈ P̅

C(Iₓ,I_{y})=B( [symbol] x,y [symbol] )

C(I_{y},Iₓ)=1−B( [symbol] x,y [symbol] )

Using the classifier directly above, a continuous valued ranking may be easily produced as R(I)=Σᵢ₌₁ⁿC(Iᵢ, I)/n. R may be the fraction of values of the training set that are “below” I based on the classifier. Thus, R may also be the empirical order statistic of I relative to the training set. The formulation above may be paired with nearly any binary classification algorithm, SVM, color histograms of annotated regions of interest, and the global severity rating (Table I) may also be used.

TABLE I

| | | | | Surrounding | | | |
|---|---|---|---|---|---|---|---|
| | | Ulcer | | Inflammation | | | |
| | Image | Sur- | | Pres./ | Sur- | Sever- | Global |
| LesionID | ID/ROI | face | Depth | Abs. | face | ity | Rating |

| | | | | | |
|---|---|---|---|---|---|
| <¼ | super- | present | <¼ | mild | mild |
| | ficial | | | | |
| ¼-½ | inter- | absent | ¼-½ | moder- | moder- |
| | mediate | | | ate | ate |
| >½ | deep | | >½ | severe | severe |

In one embodiment of the invention machine learning applications are utilized for image analysis. For example, color information in data from images may be used to isolate “non-interesting” images containing excessive food, fecal matter or air bubbles (Md. K. Bashar, K. Mori, Y. Suenaga, T. Kitasaka, and Y. Mekada. Detecting informative frames from wireless capsule endoscopic video using color and texture features. In Proc MICCAI, Springer Lecture Notes In Computer Science (LNCS), volume 5242, pages 603-611, 2008). This may be accomplished, for example, through Gabor and Discrete Wavelet based features methods. Principal Component Analysis may be used to detect motion between the image frames to create higher order motion data, and then to use the Relevance Vector Machines (RVM) method to classify contraction sequences (L. Igual, S. Segui, J. Vitria, F. Azpiroz, and P. Radeva. Eigenmotion-Based Detection of Intestinal Contractions. In Proc. CAIP, Springer Lecture Notes In Computer Science (LNCS), volume 4673, pages 293-300, 2007). Also, applying Expectation Maximization (EM) clustering on the image dataset for blood detection (S. Hwang, J. H. Oh, J. Cox, S. J. Tang, and H. F. Tibbals. Blood detection in wireless capsule endoscopy using expectation maximization clustering. In Proceedings of SPIE, pages 577-587. SPIE, 2006). And blood detection methods using for example, color spectrum transformation (Y. S. Jung, Y. H. Kim, D. H. Lee, and J. H. Kim. Active blood detection in a high resolution capsule endoscopy using color spectrum transformation. In Proc. BMEI, volume 1, pages 859-862, 2008). Methods for detecting GI organ boundaries (e.g., but not limited to, esophagus, stomach, duodenum, jejunum, ileum and colon) using, for example, energy functions (J. Lee, J. Oh, S. K. Shah, X. Yuan, and S. J. Tang. Automatic classification of digestive organs in wireless capsule endoscopy videos. In SAC '07: Proceedings of the 2007 ACM symposium on Applied computing, pages 1041-1045, New York, N.Y., USA, 2007. ACM). Use SVM to segment the GI tract boundaries (M. Coimbra, P. Campos, and J. P. S. Cunha. Topographic segmentation and transit time estimation for endoscopic capsule exams. In Proceedings of IEEE International Conference on Acoustics, Speech and Signal Processing, volume 2, pages II-II, May 2006; M. T. Coimbra and J. P. S. Cunha. Mpeg-7 visual descriptors contributions for automated feature extraction in capsule endoscopy. IEEE Transactions on Circuits and Systems for Video Technology, 16(5):628-637, May 2006). In addition, other groups have contributed to improving CE diagnosis (E. Susilo, P. Valdastri, P. Menciassi, and P. Dario. A miniaturized wireless control platform for robotic capsular endoscopy using advanced pseudokernel approach. Sensors and Actuators A: Physical, In Press, Corrected Proof, 2009; J. L. Toennies and R. J. III Webster. A wireless insufflation system for capsular endoscopes. ASME Journal of Medical Devices, accepted, 2009; P. Valdastri, A. Menciassi, and P. Dario. Transmission power requirements for novel zigbee implants in the gastrointestinal tract. Biomedical Engineering, IEEE Transactions on, 55(6):1705-1710, June 2008; M. Coimbra, P. Campos, and J. P. S. Cunha. Topographic segmentation and transit time estimation for endoscopic capsule exams. In Proceedings of IEEE International Conference on Acoustics, Speech and Signal Processing, volume 2, pages II-II, May 2006.; F Vilarino, P Spyridonos, O Pujol, J Vitria, and P Radeva. Automatic detection of intestinal juices in wireless capsule video endoscopy. In ICPR '06: Proceedings of the 18th International Conference on Pattern Recognition, pages 719-722, Washington, D.C., USA, 2006. IEEE Computer Society).

Other methods used may include motion data, and using LDA, SVM, and Hidden Markov models as well as statistical learning methods and Ordinal regression S. Bejakovic, R. Kumar, T. Dassopoulos, G. Mullin, and G. Hager. Analysis of crohns disease lesions in capsule endoscopy images. In International Conference on Robotics and Automation, ICRA, pages 2793-2798, May 2009; T. Dassopoulos, R. Kumar, S. Bejakovic, P. Rajan, S. Seshamani, G. Mullin, and G. Hager. Automated detection and assessment of crohns disease lesions in images from wireless capsule endoscopy. In Digestive Disease Week 2009, poster of distinction 2009; R. Kumar, P. Rajan, S. Bejakovic, S. Seshamani, G. Mullin, T. Dassopoulos, and G. Hager. Learning disease severity for capsule endoscopy images. In IEEE ISBI 2009, accepted, 2009; S. Seshamani, P. Rajan, R. Kumar, H. Girgis, G. Mullin, T. Dassopoulos, and G. D. Hager. A boosted registration framework for lesion matching. In Medical Image Computing and Computer Assisted Intervention (MICCAI), accepted, 2009; S. Seshamani, R. Kumar, P. Rajan, S. Bejakovic, G. Mullin, T. Dassopoulos, and G. Hager. Detecting registration failure. In IEEE ISBI 2009, accepted, 2009; OS Lin, JJ Brandabur, DB Schembre, M S Soon, and RA Kozarek. Acute symptomatic small bowel obstruction due to capsule impaction. Gastrointestinal Endoscopy, 65(4):725-728, 2007; CE Reiley, T Akinbiyi, D Burschka, D C Chang, AM Okamura, and DD Yuh. Effects of visual force feedback on robot-assisted surgical task performance. J. Thorac. Cardiovasc. Surg., 135(1):196-202, 2008; CE Reiley, H C Lin, B Varadarajan, B Vagvolgyi, S Khudanpur, DD Yuh, and GD Hager. Automatic recognition of surgical motions using statistical modeling for capturing variability. Studies in health technology and informatics, 132:396, 2008).

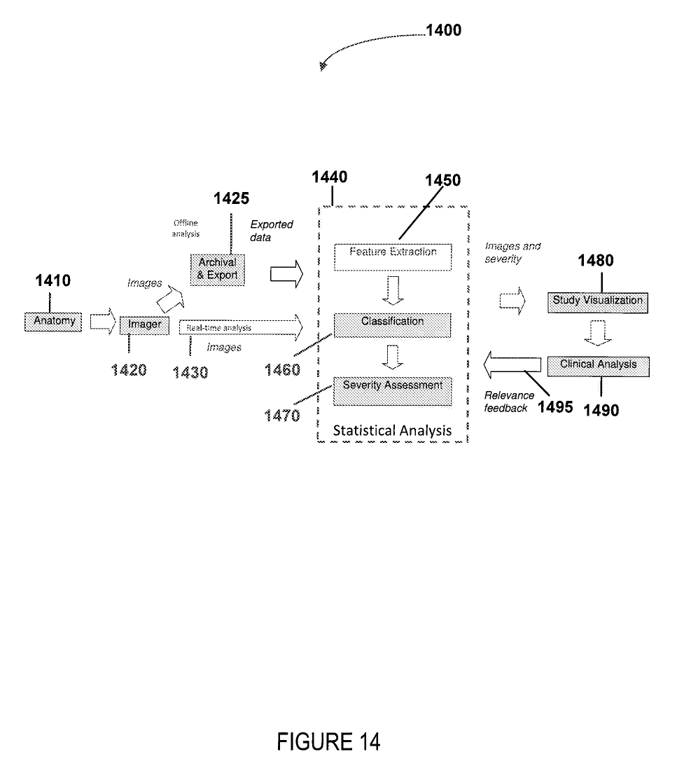

FIG. 14 depicts an illustrative information flow diagram 1400 to facilitate the description of concepts of some embodiments of the current invention. Anatomy 1410 is the starting point for the information flow as it may be the image source, such as, a GI track. An imager is shown in 1420 that takes a still image or video from anatomy 1410 through imaging tools such as 110, 120, and 130. Such imaging tools include for example, a wireless capsule endoscopy device, a flexible endoscope, a flexible borescope, a video borescope, a rigid borescope, a pipe borescope, a GRIN lens endoscope, contact hysteroscope, and/or a fibroscope.

Once the image data is taken by the imager 1420, the image data may flow to be archived for later offline analysis as shown in 1425. From 1425, the image data may flow to 1440 for statistical analysis. Alternatively, the image data could flow from the imager 1420 via 1430, as a real-time feed for statistical analysis 1440. Once the data is provided for statistical analysis in 1440, the system may perform feature extraction 1450.

Once in feature extraction 1450, feature vectors and localized descriptors may include generic descriptors such as measurements (e.g., but not limited to, color, texture, hue, saturation, intensity, energy, entropy, maximum probability, contrast, inverse difference moment, and/or correlation) color histograms (e.g., but not limited to, intensity, RBG color, and/or HSI), image statistics (e.g., but not limited to, pixel, and ROI color, intensity, and/or their gradient statistics), MPEG-7 visual descriptors (e.g., but not limited to, dominant color descriptor, edge histogram descriptor and/or its kernel weighted versions, homogeneous texture descriptor), and texture features based on Haralick statistics, as well as combinations of these descriptors. Also localized feature descriptors using spatial kernel weighting and three methods for creating kernel-weighted features may be used. Uniform grid sampling, grid sampling with multiple scales, and local mode-seeking using mean-shift may be used to allow the kernels to settle to a local maximum of a given objective function. Various objective functions may be applied, including those that seek to match generic lesion templates. Postprocessing some of these features may also be used, for example, sorting based on feature entropy or similarity to a template. Feature extraction 1450 may also be used to filter any normal or unusable data from image data which may provide only relevant frames for diagnostic purposes. Feature extraction 1450 may include removing unusable images from further consideration. Images may be considered unusable if they contain extraneous image data such as air bubbles, food, fecal matter, normal tissue, non-lesion, and/or structures.

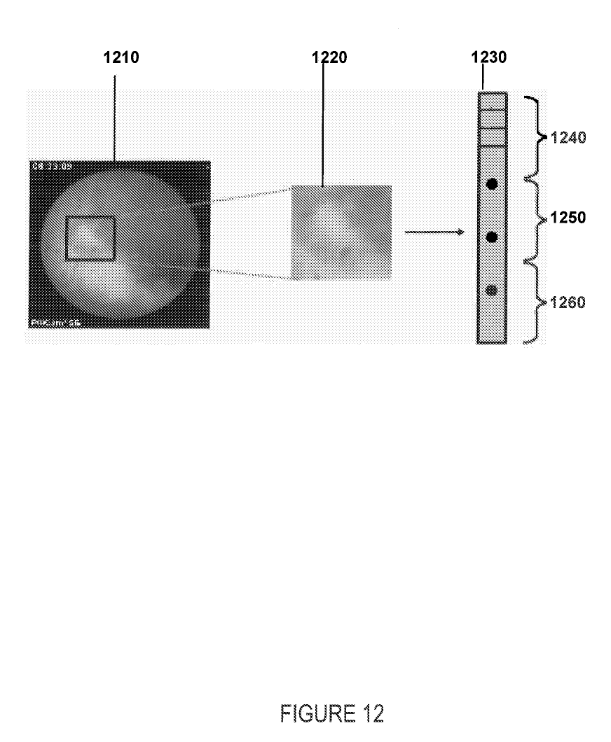

An expanded view of the feature extraction 1450 may be seen in FIG. 12, where a lesion 1220 has been detected on an image 1210 from an imager 1420, 110, 120, 130. Legion region 1220 may then be processed 1230. 1240 may include processing by an adapted dominant color descriptor (DCD) which may represent the large number of colors in an image by few representative colors which may be obtained by clustering the original colors in the image. The MPEG 7 Dominant Color Descriptor is the standard DCD. In an embodiment of the invention the DCD may differs form the MPEG-7 specification in that (i) the spatial coherency of each cluster is computed and (ii) the DCD includes the mean and the standard deviation of all colors in the image.

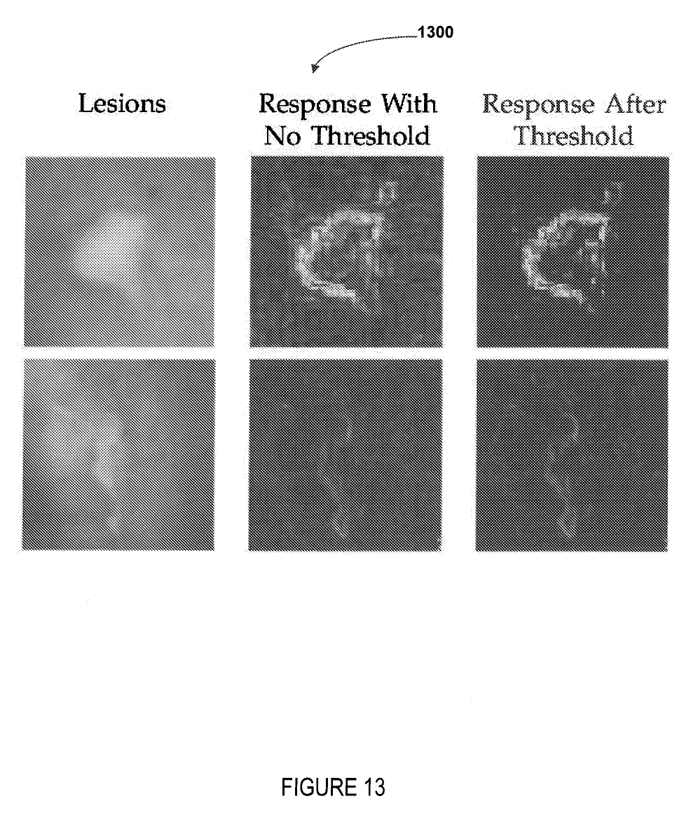

The lesion image 1220 may be processed by an adapted edge histogram descriptor (EHD) 1250 which may be an MPEG-7 descriptor that provides a spatial distribution of edges in an image. In an embodiment of the invention the MPEG-7 EHD implementation is modified by adaptive removal of weak edges. Image 1300 of FIG. 13 shows sample lesion images and the effect of using adaptive thresholds on the edge detectors responses.

The lesion image 1220 may be further processed in 1260 using image histogram statistics. This representation computes the histogram of the grayscale image and may populate the feature vector with, for example, the following values: Mean, Standard Deviation, Second moment, Third moment, Uniformity, Entropy.

From 1450, the data may flow to classification 1460. Once in classification 1460, meta-methods such as boosting and bagging methods may be used for aggregation of information from a large number of localized features. Standard techniques, e.g. voting, weighted voting, and adaboost may be used to improve classification accuracy. Temporal consistency in the classification of images may be used. For example, nearly all duplicate views of a lesion within a small temporal window. Bagging methods may be used to evaluate these sequences of images. Once an image is chosen to contain a lesion, a second classification procedure may be performed on its neighbors with, for example, parameters appropriately modified to accept positive results with weaker evidence. Sequential Bayesian analysis may also be used. Views identified to be duplicates may be presented to, for example, a clinician at the same time. Classification 1460 may include supervised machine learning and/or unsupervised machine learning. Classification 1460 may also include statistical measures, machine learning algorithms, traditional classification techniques, regression techniques, feature vectors, localized descriptors, MPEG-7 visual descriptors, edge features, color histograms, image statistics, gradient statistics, Haralick texture features, dominant color descriptors, edge histogram descriptors, homogeneous texture descriptors, spatial kernel weighting, uniform grid sampling, grid sampling with multiple scales, local mode-seeking using mean shift, generic lesion templates, linear discriminate analysis, logistic regression, K-nearest neighbors, relevance vector machines, expectation maximation, discrete wavelets, and Gabor filters. Classification 1460 may also use meta methods, boosting methods, bagging methods, voting, weighted voting, adaboost, temporal consistency, performing a second classification procedure on data neighboring said localized region of interest, and/or Bayesian analysis.

From 1460, the data may flow to severity assessment 1470. A severity of a located lesion or other attribute of interest may be calculated using a severity scale (e.g., but not limited to global severity rating shown in table I, mild, moderate, severe). The extracted features may be processed to extract feature vectors summarizing appearance, shape, and size of the attribute of interest. Additionally overall lesion severity may be more effectively computed from component indications (e.g., for example, level of inflammation, lesion size, etc.) than directly from image feature descriptions. This may be accomplished through a logistic regression (LR) that performs severity classification from attribute of interest component classifications To compute overall severity, LR, Generalized Linear Models as well as support vector regression (SVR) may be used. The assessment may include calculating a score, a rank, a structured assessment comprising of one or more categories, a structured assessment on a Likert scale, and/or a relationship with one or more other images (where the relationship may be less severe or more severe).

Prior to completing the statistical analysis an overall score based on the image data may be produced. The score may include a Lewis score, a Crohn's Disease Endoscopy index of Severity, a Simple Endoscopic Score for Crohn's Disease, a Crohn's Disease Activity Index, or another rubric based on image appearance attributes. The appearance attributes may include lesion exudates, inflammation, color, and/or texture.

Once the statistical analysis 1440 is complete, selected data, which may include a reduced set of imaging data as well as information produced during statistical analysis 1440 (e.g., but not limited to feature extraction 1450, classification 1460 of attributes of interest, and severity assessments 1470 of the attributes of interest, and score) this may be presented to a user for study at 1480. The user may analyze the information at 1490. If desired, the user may provide relevance feedback 1495 which is received by 1440 to improve future statistical analysis. Relevance feedback 1495 may be used to provide rapid retraining and re-ranking of cases, which may greatly reducing the time needed to train the system for new applications. The relevance feedback may include a change in said classification, a removal of the image from said reduced set of images, a change in an ordering of said reduced set of images, an assignment of an assessment attribute, and/or an assignment of a measurement. Once the relevance feedback is received by 1440 the system may be trained. The training may include using artificial neural networks, support vector machines, and/or linear discriminant analysis.

### Image Analysis

Analyzing CE images may require creation of higher level representations from the color, edge and texture information in the images. In one embodiment of the invention, various methods for extracting color, edge and texture features may be used including using edge features for contraction detection. Color and texture features have been used in a decision support system (M. M. Zheng, S. M. Krishnan, M. P. Tjoa; “A fusion-based clinical decision support for disease diagnosis from endoscopic images”, Computers in biology and medicine, vol. 35 pp. 259-274, 2005). Some have used MPEG-7 visual descriptors as feature vectors for topographic segmentation systems (M, Coimbra, P. Campos, J. P. Silva Cunha; “Topographic segmentation and transit time estimation for endoscopic capsule exams”, in Proc. IEEE ICASSP, 2006). While others have focused on hue, saturation and intensity (HSI) color features in their topographic segmentation systems (J. Lee, J. Oh, S. K. Shah, X. Yuan, S. J. Tang, “Automatic Classification of Digestive Organs in Wireless Capsule Endoscopy Videos”, in Proc. SAC'07, 2007).

### Extraction

One embodiment of the invention may use MPEG-7 visual descriptors and Haralick texture features. This may include MATLAB adaptation of dominant color (DCD), homogeneous texture (HTD) and edge histogram (EHD) descriptors from the MPEG-7 reference software.

### Dominant Color Descriptor (DCD)

Since Crohn's disease lesions often contain exudates and inflammation surrounding the lesion that is significantly different than normal color distributions, color space features may be used for their detection. The DCD may cluster the representative colors to provide a compact representation of the color distribution in an image. The DCD may also compute color percentages, variances, and a measure of spatial coherency.

The DCD descriptor may cluster colors in LUV space with a generalized Lloyd algorithm, for example. These clusters may be iteratively used to compute the dominant colors by, for example, minimizing the distortion within the color clusters. When the measure of distortion is high enough, the algorithm may introduce new dominant colors (clusters), up to a certain maximum (e.g., for example, 8). For example, FIG. 6 shows a sample CE image 610 and its corresponding image constructed from 6 dominant colors 620.

There may be a number of user-configurable parameters that can affect the output of the descriptor. The algorithm may iterate until the percentage change in distortion reaches a threshold (e.g., for example, 1%). Dominant color clusters may be split using a minimum distortion change (e.g., for example, 2%), and the maximum number of colors used (e.g., for example, 8. For use with CE images, we may bin the percents of dominant colors, and variances into 24̂3 bins to create feature vectors instead of using unique color and variance values in feature vectors for statistical analysis.

### Homogeneous Texture, Descriptor (HTD)

The homogeneous texture descriptor is one of three texture descriptors in the MPEG-7 standard. It may provide a “quantitative characterization of texture for similarity-based image-to-image matching.” The HTD may be computed by applying Gabor filters of different scale and orientation to an image. For reasons of efficiency, the computation may be performed in frequency space: both the image and the filters may be transformed using the Fourier transform. The Gabor filters may be chosen in such a way to divide the frequency space into 30 channels, for example, the angular direction being divided into six equal sections of 30 degrees, while the radial direction is divided into five sections on an octave scale.

The mean response and the response deviation may be calculated for each channel (each Gabor filter) in the frequency space, and these values form the features of the HTD. In addition, the HTD may also calculate the mean and deviation of the whole image in image space.

### Haralick Texture Features

Haralick texture features may be used for image classification (Haralick, R. M., K. Shanmugan, and I. Dinstein; Textural Features for Image Classification, IEEE Transactions on Systems, Man, and Cybernetics, 1973, pp. 610-621). These features may include angular moments, contrast, correlation, and entropy measures, which may be computed from a co-occurrence matrix. In one embodiment of the invention, to reduce the computational complexity, a simple one-pixel distance co-occurrence matrix may be used.

### Edge Histogram Descriptor (EHD)

The MPEG-7 edge histogram descriptor may capture the spatial distribution of edges. Four directions (0, 45, 90, and 135) and non-directional edges may be computed by subdividing the image into 16 non-overlapping blocks. Each of the 16 blocks may be further subdivided into sub-blocks, and the five edge filters are applied to each sub-block (typically 4-32 pixels). The strongest responses may then be aggregated into a histogram of edge distributions for the 16 blocks. For example, FIG. 6 shows a lesion image 630 and the corresponding combined edge responses using a sub-block size of four 640.

### EXAMPLES

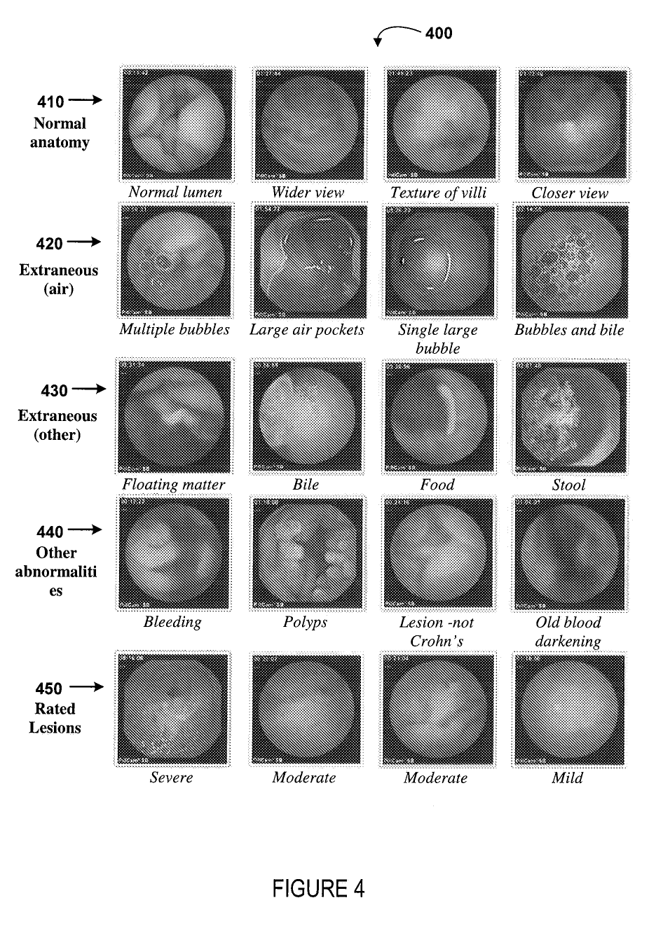

in one embodiment, support vector machines (SVM) may be used to classify CE images into lesion (L), normal tissue, and extraneous matter (food, bile, stool, air bubbles, etc). FIG. 4 depicts example normal tissue 410; air bubbles 420; floating matter, bile, food, and stool 430; abnormalities such as bleeding, polyps, non-Chrohn's lesions, darkening old blood 440; and rated lesions from severe, moderate, to mild 450. In addition to lesions other attributes of interest may include blood, bleeding, inflammation, mucosal inflammation, submucosal inflammation, discoloration, an erosion, an ulcer, stenosis, a stricture, a fistulae, a perforation, an erythema, edema, or a boundary organ

SVM has been used previously to segment the GI tract boundaries in CE images (M. Coimbra, P. Campos, J. P. Silva Cunha; “Topographic segmentation and transit time estimation for endoscopic capsule exams”, in Proc. IEEE ICASSP, 2006). SVM may use a kernel function to transform the input data into a higher dimensional space. The optimization may then estimate hyperplanes creating classes with maximum separation. One embodiment may use quadratic polynomial kernel functions using feature vectors extracted above. One embodiment may not use higher order polynomials as it may not significantly improve the results.

In one embodiment, dominant colors and variances may be binned into 24̂3 bins used as feature vectors for DCD instead of using unique color and variance values in feature vectors. Haralick features, edge histograms, and homogenous texture features may be used directly as feature vectors. Feature vectors may be cached upon computation for later use.

In one study, SVM classification was performed using only 10% of the annotated images for training. The cross-validation was performed by training using images from ninw studies, followed by classification of the images from the remaining study.

The study computed the traditional accuracy rates for each study, where

$$Accuracy = \frac{Correct\_classifications}{Total\_number \_of \_images}$$

As well as computing the sensitivity,

$$Recall = \frac{Correct\_classifications \_in \_this \_class}{Total\_anotated \_images \_in \_this \_class}$$

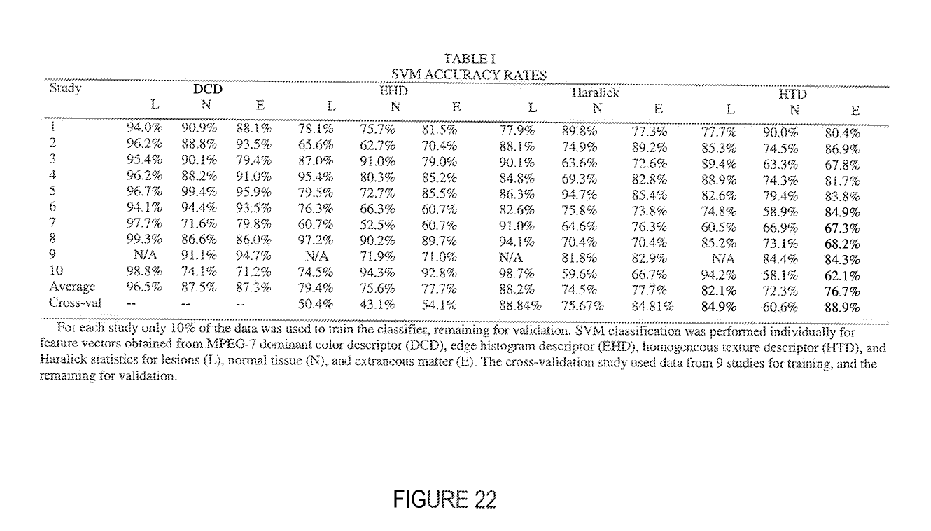

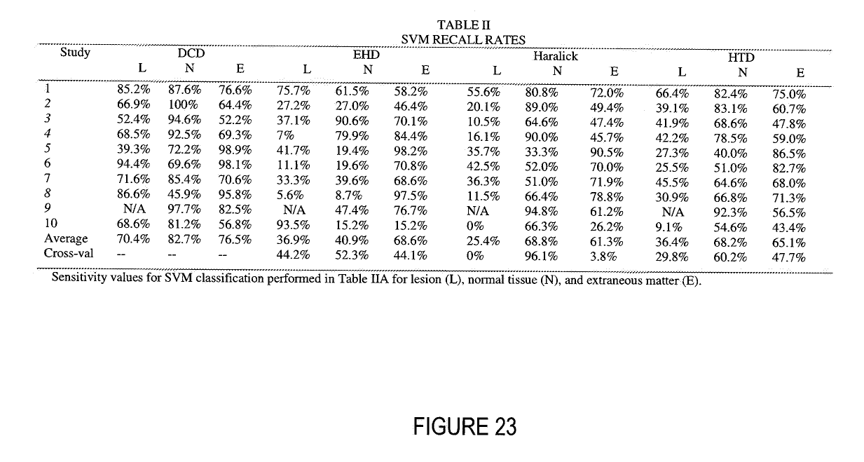

For example, SVM analysis of images from study 2 (a sample of 188 lesion images, 1231 normal images, and 266 extraneous images, for a total of 1685 images), and using a sample of 10% for training achieved classification rates of 95% for lesions, 90% for normal tissue, and 93% for extraneous matter. Over the 10 studies lesions could be detected with an accuracy rate of 96.5%, normal tissues 87.5% and extraneous matter 87.3% using dominant color information alone. FIG. 22 contains a table with the accuracy results, and FIG. 23 contains a table with the sensitivity results for the tests performed.

Cross validation was also performed using images from 9 of the studies for training, and the remaining dataset for validation. The results appear in cross-validation rows in FIG. 22 and FIG. 23. Cross-validation for DCD features was not performed. The full results appear in FIG. 22 and FIG. 23.

In one embodiment, classification based upon the color descriptor performed superior to edge, and texture based features. For lesions, this may be expected given the color information contained in exudates, the lesion, and the inflammation. The color information in the villi may also be distinct from the food, bile, bubbles, and other extraneous matter. Color information may also be less affected due to imager noise, and compression.

One embodiment may use entire CE images for computing edge and texture features. Classification performance based on edge and texture feature may suffer due use of whole images, imager limitations, fluids in the intestine, and also compression artifacts. This may be mitigated by CE protocols that require patients to control food intake before the examination, which may improve the image quality.

The variety of extraneous matter and its composition features for this class computed over entire images may not provide a true reflection of the utility of edge and texture features. In another embodiment the CE images may be segmented into individual classes (lesions, lumen, tissue, extraneous matter, and their sub-classes), and then computation of the edge and texture features may be performed. Appropriate classes (lesion, inflammation, lumen, normal tissue, food, bile, bubbles, extraneous matter, other abnormalities), instead of using entire CE images for training and validating statistical methods may be used.

### Learning Disease Severity

Classification and ranking, formulated as problems of learning a map from a set of feature to a discrete set of labels, have been applied widely in computer vision applications for face detection (P. Voila and M. Jones, “Robust real-time face diction [J],” International Journal of Computer Vision, vol. 57, no. 2, pp. 137-154, 2004), object recognition (A. Opelt, A. Pinz, M. Fussenegger, and P. Auer, “Generic Object Recognition with Boosting,” IEEE PAMI, pp. 416-431, 2006), and scene classification (R. Fergus, L. Fei-Fei, P. Perona, and A. Zisserman, “Learning object categories from google's image search,” in Proc. ICCV, 2005, pp. 1816-1823). Alternatively, ranking may be viewed as a regression problem to find a ranking function between a set of input features and a continuous range of ranks or assessment. This form has gained recent interest in many areas such as learning preferences for movies (http://www.netflixprize.com), or learning ranking functions for web pages (e.g., but not limited to, google page rank).

Learning ranking functions may require manually assigning a consistent ranking scale to a set of training data. Although the scale may be arbitrary, what is of interest is the consistent ordering of the sequence of images; a numerical scale is only one of the possible means of representing this ordering. Ordinal regression tries to learn a ranking function from a training set of partial order relationships. The learned global ranking function then seeks to respect these partial orderings while assigning a fixed rank score to each individual image or object. Both Machine learning (J. Furnkranz and E. Hullermeier, “Pairwise Preference Learning and Ranking,” Lec. Notes in Comp. Sc., pp. 145-156, 2003; R. Herbrich, T. Graepel, and K. Obermayer, Regression Models for Ordinal Data. A Machine Learning Approach, Technische Universit\ddot{}at Berlin, 1999) and content based information retrieval (S. Tong and E. Chang, “Support vector machine active learning for image retrieval,” in Proc, of 9th ACM Int. conf. on Multimedia. ACM New York, N.Y., USA, 2001, pp. 107-118) have sought to obtain mapping functions assigning preference or ranking scores. In one embodiment of the invention selective sampling techniques and SVMs with user provided sparse partial ordering in combination with image feature vectors automatically generated from a training set of images may be used.

Consider a vector of training images [symbol] ={I₁, I₂ . . . Iₙ}. A subset of [symbol] have an associated preference relationship [symbol] . Let [symbol] ={(x, y)|Iₓ [symbol] I_{y}}. Let P̅ denote the transitive closure of [symbol] . We may require that (x, x)∉P, thus disallowing inconsistent preferences. A goal may be to compute a real-valued ranking function R such that

Iₓ [symbol] I_{y}∈P [symbol] R(Iₓ)<R(I_{y})

In this embodiment, “rank” will refer to a real-valued measure on a linear scale, and “preference” will denote a comparison among objects. Given a numerical ranking on n items, O(n²) preference relationships may be generated. Likewise, given a categorization of n items into one of m bins on a scale (e.g. mild, moderate, or severe lesion), it may be possible to generate O(n²) preferences. Thus, this formulation may subsumes both scale classification and numerical regression.

In one embodiment, a preference pair [symbol] x, y [symbol] ∈ P̅ can be thought of as a pair of training examples for a binary classifier. Let us define

$$B ( p ) = \{ \begin{matrix}0 & p ∈ \overline{P} \\ 1 & otherwise\end{matrix}$$

In another embodiment, a classifier C may be trained such that for any p∈ P̅

C(Iₓ,I_{y}=B( [symbol] x,y [symbol] ) 1.

C(I_{y},Iₓ)=1−B( [symbol] x,y [symbol] ) 2.

Given such a classifier, a continuous valued ranking may be produced as

$$R ( I ) = {\sum }_{i = 1}^{n} C ( {I}_{i} , I ) / n$$

That is, R is the fraction of values of the training set that are “below” I based on the classifier. Thus, R is also the empirical order statistic of I relative to the training set. The formulation above can be paired with nearly any binary classification algorithm.

In one embodiment, SVMs may be used in combination with feature vectors extracted from the CE images. An Iₓ may be represented by a feature vector fₓ. As training examples may require pairs of images, let f_{k,j} represent the vector concatenation of fₖ and fⱼ. The training set may then consist of the set [symbol] ={<ƒ_{k,f}, 0>, <ƒ_{j,k}, 1>|(k, j)∈ P̅}. The result of performing training on [symbol] may be a classifier which, given a pair of images, may determine their relative order.

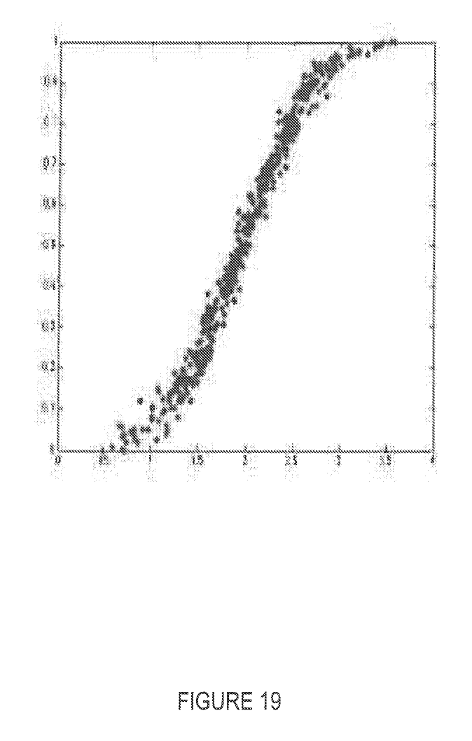

For example, random vectors in R⁴ with the following preference rule: ƒ₁ [symbol] ƒ₂ if and only if Σƒ₁<Σƒ₂. The ranking function [symbol] obtained from an SVM classifier trained on 200 samples is plotted versus Σƒ in FIG. 19. The training set included all available feature vectors, and achieved a 0% misclassification rate.

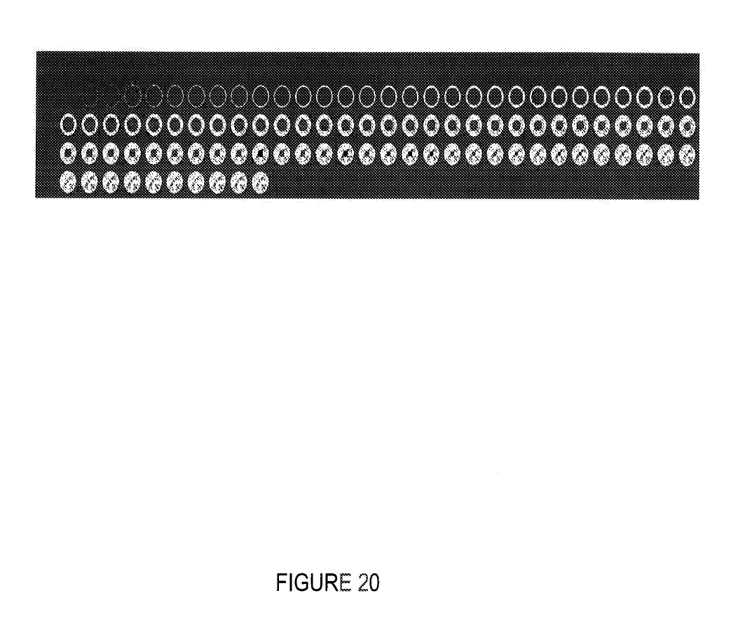

As a second example, consider a set of 100 synthetic images of disks of varying thickness an example shown in FIG. 20. Each image may be 131×131 and gray scale, with the disc representing the only non-zero pixels, consecutive images differing by 0.5 pixels in disc thickness. For images Ii and Ij, the underlying ranking function is thickness(i)<thickness(j)≡i [symbol] j. Using, for example, a 10 bin intensity histograms as the feature vector, a SVM classifier using radial basis functions produces a ranking function [symbol] that correctly orders (0% misclassification) the discs FIG. 20 using only O(n) pairwise relationships.

### Embodiment

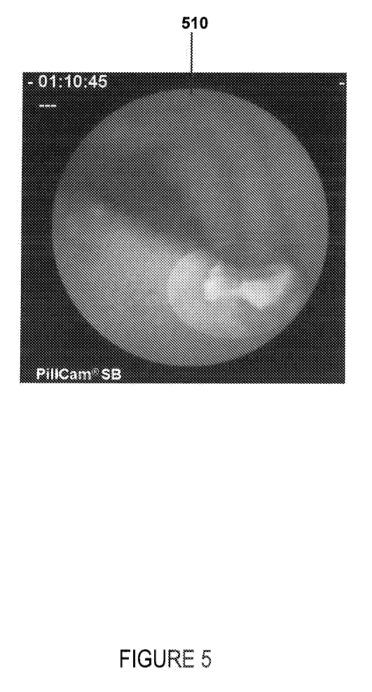

In one embodiment, lesions as well as data for other classes for interest may be selected and assigned a global ranking (e.g., for example, mild, moderate, or severe) based upon the size, and severity of lesion and any surrounding inflammation, for example. Lesions may be ranked into three categories: mild, moderate or severe disease. FIG. 5, 510 shows a typical Crohn's disease lesion with the lesion highlighted. As a lesion may appear in several images, data representing 50 seconds, for example, of recording time around the selected image frame may also be reviewed, annotated, and exported as part of a sequence. In addition, a number of extra image sequences not containing lesions may be exported as background data for training of statistical methods.

Global lesion ranking may be used to generate the required preference relationships. For example, over 188,000 pairwise relationships may be possible in a dataset of 600 lesion image frames that have been assigned a global ranking of mild, moderate or severe by a clinician, assuming mild<moderate<severe. In one embodiment, a small number of images may be used to initiate training, and an additional number to iterate for improvement of the ranking function. Previous work on machine learning has generally made use of some combination of color and texture features. SIFT is not very suitable for our wireless endoscopy images, due to lack of sufficient number of SIFT features in these images (D. G. Lowe, “Object recognition from local scale-invariant features,” in Proc. ICCV. Kerkyra, Greece, 1999, vol. 2, pp. 1150-1157). A variety of feature vectors including, for example edge, color, and texture features, MPEG-7 visual descriptors, and hue, saturation and intensity features have been published specifically for analysis of wireless capsule endoscopy images (Y. Liu, D. Zhang, G. Lu, and W. Y. Ma., “A survey of content based image retrieval with high-level semantics,” Pattern Recognition, vol. 40, no. 1, pp. 262-282, 2007; M. Coimbra, P. Campos, and JPS Cunha, “Topographic Segmentation and Transit Time Estimation for Endoscopic Capsule Exams,” in Proc. ICASSP, 2006, vol. 2; Jeongkyu Lee, JungHwan Oh, Subodh Kumar Shah, Xiaohui Yuan, and Shou Jiang Tang, “Automatic classification of digestive organs in wireless capsule endoscopy videos,” in SAC07, 2007). In one embodiment, improvement of accuracy of the ranking function may be shown with increasing number of pairwise preferences.

In another embodiment, on n±100 images, starting with only O(n) training relationships, and SVM classifier using radial basis functions as before, we obtain only O(n2) mismatches using the generated ranking function R after the first iteration. A mismatch is any pair of images where R(Ix)< or >R(Iy) and Ix> or <Iy The number of mismatches drops exponentially over 4 iterations where the training set is increased by m=max(1000, mismatches) pairwise relationships.

TABLE II

| Metric | Iter. 2 | Iter. 3 | Iter. 4 |
|---|---|---|---|
| Mean | 0.1133 | 0.0182 | 0.0024 |
| Std. Dev | 0.3055 | 0.0915 | 0.0106 |

| Metric | Iter. 1 | Iter. 2 | Iter. 3 | Iter. 4 |
|---|---|---|---|---|
| Training size | 100 | 1100 | 1972 | 2116 |
| mismatches | 1286 | 436 | 77 | 3 |

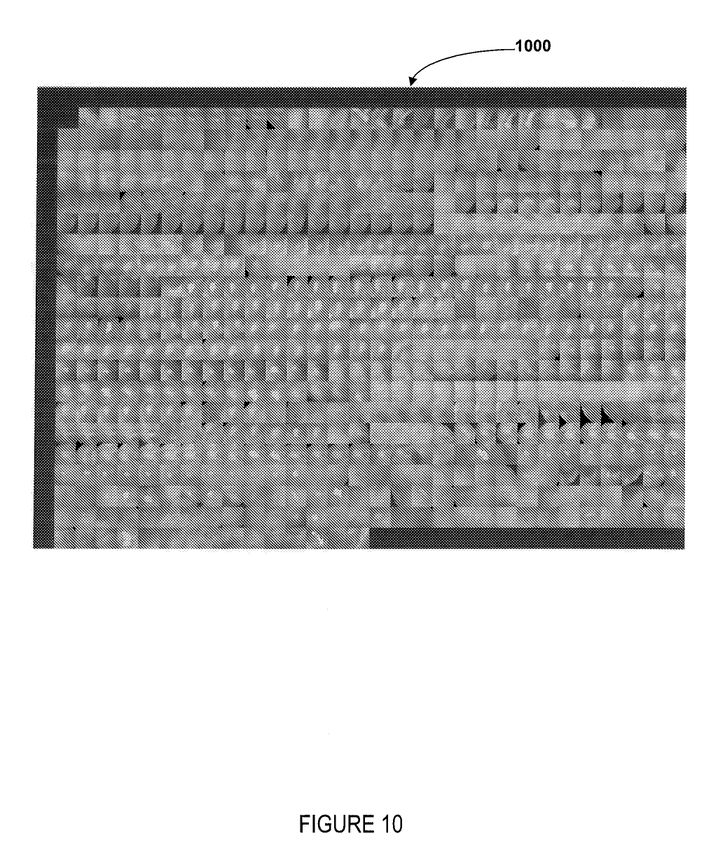

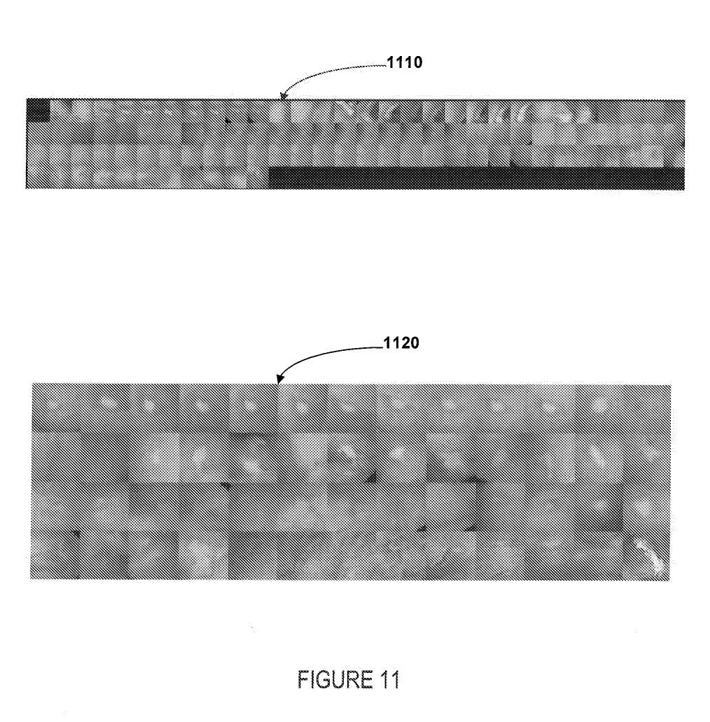

FIG. 11, 1110 and 1120 show an example of a ranked images data set. Table II shows, for example, changes in ranks for images, and number of mismatches during each iteration. Both the mean and standard deviation of rank change for individual images decreases monotonously over successive iterations. Table II also shows the decreasing number of mismatches over successive iterations. The ranking function may converge after a few iterations, with the changes in rank becoming smaller closer to the convergence. FIG. 10, 1000 depicts 500 lesion images that may be similarly ranked.

### Boosted Registration Framework for Lesion Matching

Minimally invasive diagnostic imaging methods such as flexible endoscopy, and wireless capsule endoscopy (CE) often present multiple views of the same anatomy. Redundancy and duplication issues are particularly severe in the case of CE, where peristalsis propulsion may lead to duplicate information for several minutes of imaging. This may be difficult to detect, since each individual image captures only a small portion of anatomical surface due to limited working distance of these devices, providing relatively little spatial context. Given the relatively large anatomical surfaces (e.g. the GI tract) to be inspected, it is important to identify duplicate information as well as present all available views of anatomical and disease views to the clinician for improving consistency, efficiency and accuracy of diagnosis and assessment.

The problem of image duplication has been commonly formulated as a detection problem Taylor, C. J., Cooper, D. H., Graham, J.: Training models of shape from sets of examples. In: In Proc. British Machine Vision Conference, Springer-Verlag (1992) 9-18 where a classifier is trained to learn the visual properties of the chosen object category (i.e. lesions). This process typically requires feature extraction to generate a low dimensional representation of image content, followed by classifier training to distinguish the desired object model(s) (Viola, P., Jones, M.: Rapid object detection using a boosted cascade of simple features. In: CVPR '01: Proceedings of the 2001 IEEE Computer Society Conference on Computer Vision and Pattern Recognition (CVPR '01). (2001) 511-518). For CE, appearance modelling has been used for blood detection (Jung, Y. S., Kim, Y. H., Lee, D. H., Kim, J. H.: Active blood detection in a high resolution capsule endoscopy using color spectrum transformation. In: BMEI '08: Proceedings of the 2008 International Conference on BioMedical Engineering and Informatics, Washington, D.C., USA, IEEE Computer Society (2008) 859-862; Hwang, S., Oh, J., Cox, J., Tang, S. J., Tibbals, H. F.: Blood detection in wireless capsule endoscopy using expectation maximization clustering. Volume 6144., SPIE (2006) 61441P; Li, B., Meng, M. Q. H.: Computer-based detection of bleeding and ulcer in wireless capsule endoscopy images by chromaticity moments. Comput. Biol. Med. 39(2) (2009) 141-147) topographic segmentation (Cunha, J., Coimbra, M, Campos, P., Soares, J.: Automated topographic segmentation and transit time estimation in endoscopic capsule exams. 27(1) (January 2008) 19-27) and lesion classification (Bejakovic, S., Kumar, R., Dassopoulos, T., Mullin, G., Hager, G.: Analysis of crohn's disease lesions in capsule endoscopy images. In: IEEE ICRA. (2009 (accepted)). However, generic detection may be different than matching an instance of a model to another instance.

In one embodiment of the invention, the problem of detecting repetitive lesions may be addressed as a registration and matching problem. A registration method may evaluate an objective function or similarity metric to determine a location in the target image (e.g., for example, a second view) where a reference view (e.g., for example, a lesion) occurs. Once a potential registration is computed, a decision function may be applied to determine the validity of the match. In one embodiment of the invention a trained statistical classifier is used that makes a decision based on the quality of a match between two regions of interest (ROIs) or views of the same lesion, rather than the appearance of the features representing an individual ROI.

Decision functions for registration and matching have traditionally been designed by thresholding various similarity metrics. The work of Szeliski et al (Szeliski, R.: Prediction error as a quality metric for motion and stereo. In: ICCV '99: Proceedings of the International Conference on Computer Vision-Volume 2, Washington, D.C., USA, IEEE Computer Society (1999) 781) and Stewart et al (Yang, G., Stewart, C., Sofka, M., Tsai, C. L.: Registration of challenging image pairs: Initialization, estimation, and decision. Pattern Analysis and Machine Intelligence, IEEE Transactions on) are examples of such problem formulations. In many cases, a single, unique global threshold may not exist; but, the determination of an adaptive threshold is a challenging problem. Alternatively, Chen et al (Chen, X., Chain, T. J.: Learning feature distance measures for image correspondences. In: CVPR '05: Proceedings of the 2005 IEEE Computer Society Conference on Computer Vision and Pattern Recognition (CVPR'05)—Volume 2, Washington, D.C., USA, IEEE Computer Society (2005) 560-567) introduce a new feature vector that represents images using an extracted feature set. However, this approach still requires the same similarity metric across the entire feature set. By contrast, we present a generalizable framework that incorporates multiple matching algorithms, a classification method trained from registration data, and a regression based ranking system to choose the highest quality registration.

### Boosted Registration

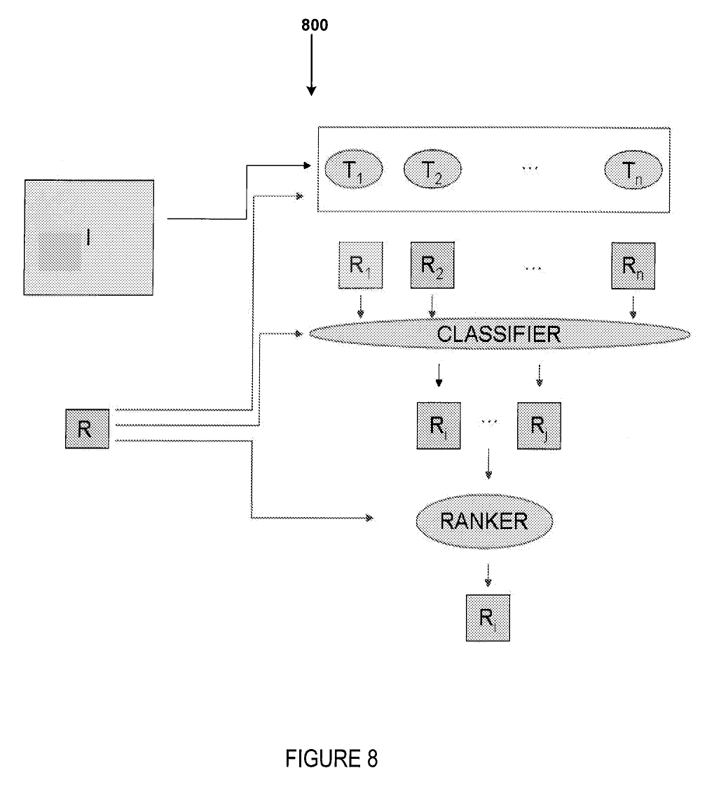

The objective function for a registration method may be based upon the invariant properties of the data to be registered. For example, histograms are invariant to rotation, whereas pixel based methods are generally not. Feature based methods may be less affected by changes in illumination and scale. Due to large variation in these invariance properties within endoscopic studies, a single registration method may not be appropriate for registration of this type of data. Instead, one embodiment may use multiple independent registration methods, each may be more accurate in a different subset of the data, and a global decision function that may use a range of similarity metrics to estimate a valid match. Multiple acceptable estimates are may be ranked using a ranking function to determine the best result. FIG. 8, 800 depicts an example information flow in an exemplarily embodiment. For example, given an ROI Rᵢ in an image i and a target image Iⱼ the registration function T(Rᵢ, Iⱼ) [symbol] Rⱼ, maps Rᵢ to Rⱼ. The similarity metric relating the visual properties of Rᵢ and Rⱼ may be defined as d(Rᵢ, Rⱼ). Using a set of registration functions [symbol] =Tᵢ(R, I); i=1 . . . n and estimated or annotated ROIs R₁′ . . . Rₙ′, the decision function D may determine which estimates are correct matches.

$$D ( {R}_{i} , {R}_{j}^{′} ) = \{ \begin{matrix}1 , & if d ( {R}_{i} , {R}_{j}^{′} ) < γ \\ - 1 & otherwise\end{matrix}$$

### Decision Function Design:

In one embodiment, the decision function may be designed by selection of a set of metrics to represent a registration and application of a thresholding function on each metric to qualify matches. Although false positive rates can be minimized by such a method, the overall retrieval rate may be bounded by the recall rate of the most sensitive metric. An integrated classifier that distinguishes registrations based on a feature representation populated by a wide range of metrics may be likely to outperform such thresholding. In one exemplarily embodiment, an ROI R, the following notation may be used in representing appearance features. Starting with pixel based features. The intensity band of the image may be denoted as R₁. The Jacobian of the image may be denoted R_{J}=[Rₓ, R_{y}] where Rₓ and R_{y} may be the vectors of spatial derivatives at all image pixels. Condition numbers and the smallest eigen values of the Jacobian may be denoted as R_{JC} and R_{JE} respectively. The Laplacian of the image is denoted as R_{LAP}. Following this, histogram based features may be defined as: R_{RGBH}, R_{WH} and R_{WCH} for RGB histograms, gaussian weighted intensity histograms and gaussian weighted color histograms respectively. Also, MPEG-7 features: R_{EHD} (Edge Histogram Descriptors), R_{Har} (Haralick Texture descriptors) and R_{HTD} (Homogeneous Texture Descriptors). Given two images Iₐ and I_{b} where A is an ROI in Iₐ with center x and B is an ROI in R_{b}, a feature vector may be generated for a pair of regions A and B populated with the metrics shown in table III, for example. The decision function may then be trained to distinguish between correct and incorrect matches using any standard classification method. We use support vector machines (SVM) (Vapnik, V. N.: The nature of statistical learning theory. Springer-Verlag New York, Inc., New York, N.Y., USA (1995)) in our experiments.

TABLE III

| Metric Name | Formula |
|---|---|
| RMS (rms) | $\sqrt{( \frac{1}{n} {\sum }_{k} {( {A}_{I} - {B}_{I} )}^{2} )}$ |
| RMS Shuffle | $\sqrt{( \frac{1}{n} {\sum }_{k} shuffle ( {A}_{I} , {B}_{I} ) )}$ |
| Ratio of Condition Numbers | min(A_{JC}, B_{JC})/max(A_{JC}, B_{JC}) |
| Ratio of Smallest Eigen Values | min(A_{JE}, B_{JE})/max(A_{JE}, B_{JE}) |
| Laplacian Shuffle Distance | shuffle(A_{LAP}, B_{LAP}) |
| Weighted Histogram Bhattacharya | sqrt(A_{WH}, B_{WH}) |
| Distance | |
| RGB Histogram Bhattacharya Distance | sqrt(A_{RGB} − B_{RGB}) |
| Edge Histogram Manhattan Distance | Σ(A_{EHD} − B_{EHD}) |
| Haralick Descriptor Canberra Distance | $\sum \frac{\vert {A}_{Har} - {B}_{Har} \vert}{\vert {A}_{Har} + {B}_{Har} \vert}$ |
| HTD Shuffle Distance | shuffle(A_{WTD}, B_{WTD}) |
| Forward Backward check | \|x − T(I_{b}, Iₐ, T(Iₐ, I_{b}, x))\| |

### The Ranking Function:

In yet another embodiment of the invention, the registration selection may be treated as an ordinal regression problem (Herbrich, R., Graepel, T., Obermayer, K.: Regression Models for Ordinal Data: A Machine Learning Approach. Technische Universität Berlin (1999)). Given a feature set corresponding to correctly classified registrations, F={ƒ₁, . . . , ƒ_{N}} and a set of N distances from the true registrations; a set of preference relationships may form between the elements of F. The set of preference pairs P may be defined as, P={(x, y)|ƒₓ [symbol] ƒ_{y}}. In one embodiment, a continuous real-valued ranking function K is computed such that, ƒₓ [symbol] ƒ_{y}∈P [symbol] K(ƒₓ) [symbol] K(ƒ_{y}). A preference pair (x, y)∈P may be considered a pair of training examples for a standard binary classifier. A binary classifier C may be trained such that,

$$C ( {F}_{x} , {F}_{y} ) = \{ \begin{matrix}0 , & if ( x , y ) ∈ P \\ 1 & otherwise\end{matrix} \\ C ( {F}_{y} , {F}_{x} ) = 1 - C ( {F}_{x} , {F}_{y} )$$

Given such a classifier, the rank may be computed as, K(F)=Σᵢ₌₁ⁿ C(F, Fᵢ)/n where K may be the fraction of the training set that are less preferred to F based on the classifier. Thus, for example, K orders F relative to the training set. Support Vector Machines (SVM) may be used for binary classification. Let ƒₓ represent the metrics or features of registration and ƒ_{i,j} represent the vector concatenation of ƒᵢ and ƒⱼ. The training set, Train={<ƒ_{i,j}, 0>, <ƒ_{j,i}, 1≧|(i, j)∈P} may be used to train an SVM. For classification, each vector may be paired in the test set with all the vectors in the training set and the empirical order statistics K(F) described above may be used for enumerating the rank.

### Training Data

Given an ROI R and a set of images [symbol] =Iᵢ: i=1 . . . N, one embodiment may build a dataset of pairs of images representing correct and incorrect matches of a global registration. First computed may be the correct location of the center of the corresponding ROI in [symbol] Through manual selection followed by a local optimization, for example. This set of locations may be denoted as X=Xᵢ: i=1 . . . N. Next, any global registration method T may be selected and applied between R and each image in the set [symbol] to generate a set of estimated ROI center locations X′=Xᵢ′: i=1 . . . N and pairs [symbol] ={R, Rᵢ: i=1 . . . }. The pairs may be designated a classification y (correct or incorrect matches) by thresholding on the L2 between Xᵢ and Xᵢ′, for example. This may be referenced as the ground truth distance. The training set T may contain all registered pairs and their associated classifications.

### Experiments

One embodiment of the invention was tested using a CE study database which contained selected annotated images containing Crohn's Disease (CD) lesions manually selected by our clinical collaborators. These images provided the ROIs for our experiments. A lesion may occur in several neighboring images, and these selected frames form a lesion set. FIG. 9, 910 shows an example of a lesion set. In these experiments, 150×150 pixel ROIs were selected. Various lesion sets contained between 2 and 25 image frames. Registration pairs were then generated for every ROI in the lesion set, totaling 266 registration pairs.

In this embodiment, registration methods spanning the range of standard techniques for 2d registration were used. These include SIFT feature matching, a mutual information optimization, weighted histograms (grayscale and color) and template matching. For each of these methods, a registration to estimate a registered location was performed, resulting in a total of 1330 estimates (5 registration methods per ROI-image pair). The ground truth for these estimates was determined by thresholding the L2 distance described above, and it contains 581 correct (positive examples) and 749 incorrect (negative examples) registrations.

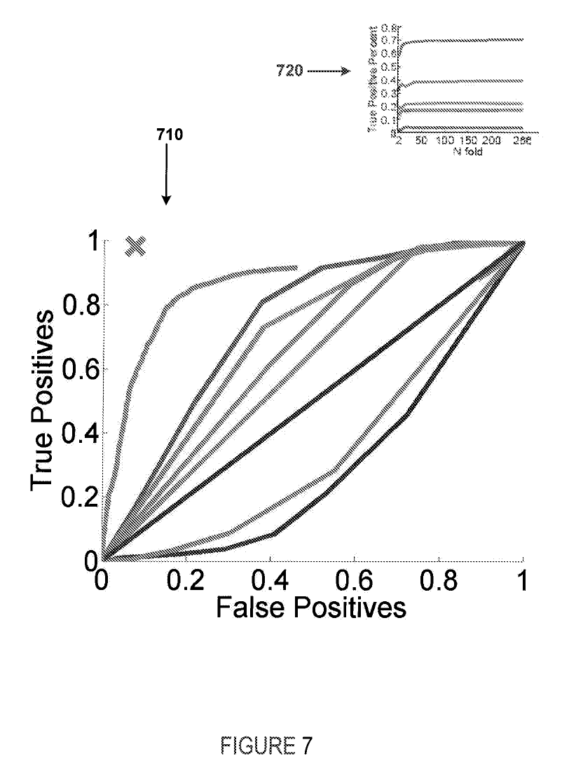

In this embodiment, for every registration estimate, we compute the registered ROI for the training pair. The feature vector representing this registration estimate is then computed as described in section 2. We then train the decision function using all registration pairs in the dataset. The performance of this integrated classifier was evaluated using a 10-fold cross-validation. FIG. 7 shows the result on training data, including comparison with the ROC curves of individual metrics used for feature generation. The true positive rate is 96 percent and the false negative rate is 8 percent.

In this embodiment, for n registrations, a total of nC₂ preference pairs can be generated. A subset of this data may be used as the input to the ranking model. Features used to generate a training pair may include the difference between Edge Histogram descriptors and the difference between the dominant color descriptors. Training may be initiated with a random selection of n=200. This estimate may then be improved by iteration and addition of preference pairs at every step. Training may be conducted using an SVM model with a radial basis kernel. At each iteration, the dataset may be divided into training and test sets. A classifier may be trained and preference relationships may be predicted by classifying vectors paired with all training vectors, Relative ranks within each set may be determined and pair mismatch rates may then be calculated. A mismatch may be any pair of registrations where K(F)>K(F_{y}) and Fₓ<F_{y} or K(Fₓ)<K(F_{y}) and Fₓ>F_{y}. The training mis-classification rate may be the percentage of contradictions between the true and predicted preference relationships in the training set. Table IV shows an example rank metrics for each iteration.

TABLE IV

| | Iter1 | Iter2 | Iter3 | Iter4 | Iter5 | Iter6 | Iter7 | Iter8 |
|---|---|---|---|---|---|---|---|---|
| No: of pairs | 300 | 600 | 900 | 1200 | 1500 | 1800 | 2100 | 2400 |
| Train mis-classification rate | 0.001 | 0.014 | 0.016 | 0.015 | 0.018 | 0.017 | 0.017 | 0.017 |
| Train pair mismatch rate | 0.16 | 0.18 | 0.17 | 0.16 | 0.16 | 0.16 | 0.16 | 0.15 |
| Test pair mismatch rate | 0.32 | 0.38 | 0.32 | 0.26 | 0.38 | 0.32 | 0.35 | 0.27 |
| Test rank mean | 0.53 | 0.69 | 0.55 | 00.35 | 0.69 | 0.55 | 0.61 | 0.44 |
| Test rank std dev | 0.14 | 0.15 | 0.20 | 0.28 | 0.19 | 0.23 | 0.21 | 0.29 |

In one embodiment, the boosted registration framework may be applied to all image pairs. For each pair, all 5 registration methods, for example, may be applied to estimate matching ROIs. For example, the first row of table V shows the number of correct registrations evaluated using the ground truth distance. Features may then be extracted for all registrations and the integrated classifier, as described above, may be applied. A leave one out cross-validation may be performed for each ROI-image pair. The second row of table V shows the number of matches that the classifier validates as correct. Finally, the last row in sample table V shows the number of true positives (i.e., the number of correctly classified matches that are consistent with the ground truth classification). The last column in sample table V shows the performance of the boosted registration. The number of registrations retrieved by the boosted framework may be greater than any single registration method. A range of n-fold validations may be performed on the same dataset for n ranging from 2−(the number of image pairs) (where n=2 divides the set into two halves and n=number of image pairs may be the leave one out validation). FIG. 7, 720 shows an example of the percentage of true positives retrieved (which is the ratio of true positives of the boosted registration to the number of correct ground truth classifications) by each individual registration method and the boosted classifier (e.g., cyan). The boosted registration may outperforms many other methods. FIG. 7, 710 show the ROC Curves of all metrics used individually overlaid with the integrated classifier (Green X).

TABLE V

| | Tem- | | | | | |
|---|---|---|---|---|---|---|
| | plate | | | Intensity | HSV | Boosted |
| | Match- | | Mutual | Weighted | Weighted | Registra- |
| Type | ing | Sift | Info | Histogram | Histogram | tion |
| Ground | 165 | 122 | 54 | 111 | 129 | 266 |
| Truth | | | | | | |
| Classi- | 129 | 62 | 25 | 75 | 77 | 188 |
| fier | | | | | | |
| True | 106 | 59 | 10 | 46 | 47 | 188 |
| Positives | | | | | | |

In one embodiment of the invention, a boosted registration framework for the matching of lesions in capsule endoscopic video may be used. This generalized approach may incorporate multiple independent optimizers and an integrated classifier combined with a trained ranker to select the best correct match from all registration results. This method may outperform the use of any one single registration method. In another embodiment, this may be extended to hierarchical sampling where a global registration estimate may be computed without explicit application of any particular optimizer.

### A Meta Method for Image Matching: Two Applications

Image registration involves estimation of a transformation that relates pixels or voxels in one image with another one. There are generally two types of image registration methods: image based (direct) and feature based. Image based methods (Simon Baker, Ralph Gross, and lain Matthews, “Lucas-kanade 20 years on: A unifying framework: Part 4,” International Journal of Computer Vision, vol. 56, pp. 221-255, 2004; Gregory D. Hager and Peter N. Belhumeur, “Efficient region tracking with parametric models of geometry and illumination,” IEEE Transactions on Pattern Analysis and Machine Intelligence, vol. 20, pp. 1025-1039, 1998) utilize every pixel or voxel in the image to compute the registration whereas feature based methods (Ali Can, Charles V. Stewart, Badrinath Roysam, and Howard L. Tanenbaum, “A feature-based technique for joint linear estimation of high-order image-to-mosaic transformations: Mosaicing the curved human retina,” IEEE Transactions on Pattern Analysis and Machine Intelligence, vol. 24, no. 3, pp. 412-419, 2002) use a sparse set of corresponding image features for this. Both methods use a matching function or matcher that quantifies the amount of similarity between images for an estimated transformation. Examples of matchers include: Sum of Squared Differences (SSD), Normalized Cross Correlation (NCC), Mutual Information (MI), Histogram Matchers, etc.

Each matcher has a set of properties that make it well suited for registration of certain types of images. For example, Normalized Cross Correlation can account for changes in illumination between images, histogram based matchers are invariant to changes in rotation between images, and so on. These properties are typically referred to as invariance properties (Remco C. Veltkamp, “Shape matching: Similarity measures and algorithms,” in SMI '01: Proceedings of the International Conference on Shape Modeling & Applications, Washington, D.C., USA, 2001, p. 188, IEEE Computer Society). Matchers are typically specialized to deal with only a small set of properties in order to balance the trade-off between robustness to invariance and accuracy.

Many applications contain data that require only a few known properties to be accounted for. In such cases, it is easy to select the matcher that has the appropriate invariance property. However, the properties of medical image data are usually unpredictable and this makes it difficult to select a specific matcher. For example, 910 of FIG. 9 shows a sequence of images from a capsule endoscope containing the same anatomical region of interest. By observing just a few images from this dataset, we can already note variations in illumination, scale and orientation. In the case where we are interested in registration of anatomical regions across all these invariance properties, selecting a robust and accurate matcher for the task is very difficult.

One approach to addressing this problem is to utilize a matching function that combines matchers with different invariance properties. For example, Wu et al. (Jue Wu and Albert Chung, “Multi-modal brain image registration based on wavelet transform using sad and mi,” in Proc. Int'l Workshop on Medical Imaging and Augmented Reality. 2004, vol. 3150, pp. 270-277, Springer) use the Sum of Absolute Differences (SAD) and Mutual Information (MI) for multi-modal brain image registration. Yang et al. (Gehua Yang and Charles V. Stewart, “Covariance-driven mosaic formation from sparsely-overlapping image sets with application to retinal image mosaicing,” in Proc. IEEE Conf. Computer Vision and Pattern Recognition, 2004, pp. 804-810) use a feature based method where covariance matrices of transformation parameters and the Mahalanobis distance between the feature sets are used for matching retinal images. More recently, Atasoy et al. (Selen Atasoy, Ben Glocker, Stamatia Giannarou, Diana Mateus, Alexander Meining, Guang-Zhong Yang, and Nassir Navab, “Probabilistic region matching in narrow-band endoscopy for targeted optical biopsy,” in Proc. Int'l Conf. on Medical Image Computing and Computer Assisted Intervention, 2009, pp. 499-506) propose an MRF-based matching technique that incorporates region based similarities and spatial correlations of neighboring regions, applied to Narrow-Band Endoscopy for Targeted Optical Biopsy. However, for a dataset with several properties to account for, developing an appropriate matching function is a complex task.

Metamatching (S. Seshamani, P. Rajan, R. Kumar, H. Girgis, G. Mullin, T. Dassopoulos, and G. D. Hager, “A meta registration framework for lesion matching,” in Int'l Conf. on Medical Image Computing and Computer Assisted Intervention, 2009, pp. 582-589) offers an alternative approach to addressing this problem. A metamatching system consists of a set of matchers and a decision function. Given a pair of images, each matcher estimates corresponding regions between the two images. The decision function then determines if any of these estimates contain similar regions (either visually and/or semantically, depending on the task). This type of approach may be generic enough to allow for simple matching methods with various invariance properties to be considered. In addition, it may also increase the chance of locating matching regions between images. However, this method relies on a decision function that can accurately decide when two regions match.

In one embodiment of the invention, a trained binary classifier as a decision function is used for determining when two images match. A thorough comparison of the use of standard classifiers: Nearest neighbors, SVMs, LDA and Boosting with several types of region descriptors may be performed. In another embodiment, a metamatching framework based on a set of simple matchers and these trained decision functions may be used. The strength of the embodiment is demonstrated with registration of complex medical datasets using very simple matchers (such as template matching, SIFT, etc), Applications considered may include Crohn's Disease (CD) lesion matching in capsule endoscopy and video mosaicking in hysteroscopy. In the first application, the embodiment may perform global registration and design a decision function that may distinguish between semantically similar and dissimilar images of lesions. In the second application, the embodiment may considers the scenario of finer registrations for video mosaicking and the ability to train a decision function that can distinguish between correct and incorrect matches at a pixel level, for example.

The design of a decision function may be based on a measure (or set of measures) that quantifies how well an image matches another image. This type of measure may be called a similarity metric (Hugh Osborne and Derek Bridge, “Similarity metrics: A formal unification of cardinal and non-cardinal similarity measures,” in Proc. Int'l Conf. on Case-Based Reasoning. 1997, pp. 235-244, Springer). Matching functions (e.g., for example, NCC, Mutual information, etc) are often used as similarity metrics. For example, Szeliski (Richard Szeliski, “Prediction error as a quality metric for motion and stereo,” in Proc. IEEE Int'l Conf. on Computer Vision, 1999, pp. 781-788) uses the RMS (and some of its variants) for error prediction in motion estimation. Kybic et al. (Jan Kybic and Daniel Smutek, “Image registration accuracy estimation without ground truth using bootstrap,” in Int'l Workshop on Computer Vision Approaches to Medical Image Analysis, 2006, pp. 61-72) introduce the idea of bootstrap-based uncertainty metrics to evaluate the quality of pixel-based image registration. Yang et al. (Gehua Yang, Charles V. Stewart, Michal Sofka, and Chia-Ling Tsai, “Registration of challenging image pairs: Initialization, estimation, and decision,” IEEE Transactions on Pattern Analysis and Machine Intelligence, vol. 29, no. 11, pp. 1973-1989, 2007) use a generalized bootstrap ICP algorithm to align images and apply three types of metrics: an accuracy estimate, a stability estimate and consistency of registration estimate. Here, a match is qualified as correct only if all three estimates fall below a certain threshold. Adaptive thresholding techniques (X-T Dai, L Lu, and G Hager, “Real-time video mosaicing with adaptive parameterized warping,” in IEEE Conf. Computer Vision and Pattern Recognition, 2001, Demo Program) have also been proposed for performing registration qualification. All these methods work as threshold based binary classifiers. One disadvantage of this approach may be that threshold selection is a manual process. Also, in the case where several metrics are used, a hard voting scheme is often used, where a match is qualified as correct only if it satisfies threshold conditions of all metrics. This may lead to the problem of either large numbers of false negatives (i.e., correct matches which are qualified as wrong) if the thresholding is too strong or false positives (incorrect matches that are qualified as correct).

Recently the area of distance metric learning (Liu Yang and Rong Jin, “Distance metric learning: A comprehensive survey,” Tech. Rep., 2006) has shown a considerable amount of interest in applying learning for the design of pairwise matching decision functions. Unlike threshold based techniques, the metric learning problem may involve selection of a distance model and learning (either supervised or unsupervised) parameters that distinguish between similar and dissimilar pairs of points. One problem may be supervised distance metric learning, where the decision function is trained based on examples of similar and dissimilar pairs of images.

There may be two broad groups of supervised metric learning, global metric learning and local metric learning. Global methods may consider a set of data points in a feature space and model the distance function as a Mahalanobis distance between points. Then, using points whose pairwise similarity may be known, the covariance matrix (of the Mahalanobis distance) may be learned using either convex optimization techniques (Eric P. Xing, Andrew Y. Ng, Michael I. Jordan, and Stuart Russell, “Distance metric learning, with application to clustering with side information,” in Advances in Neural Information Processing Systems. 2002, pp. 505-512, MIT Press) or probabilistic approaches (Liu Yang and Rong Jin, “Distance metric learning: A comprehensive survey,” Tech. Rep., 2006). Local distance metrics (Liu Yang, Rong Jin, Lily Mummert, Rahul Sukthankar, Adam Goode, Bin Zheng, Steven C. H. Hoi, and Mahadev Satyanarayanan, “A boosting framework for visuality-preserving distance metric learning and its application to medical image retrieval,” IEEE Trans. Pattern Anal. Mach. Intell., vol. 32, no. 1, pp. 30-44, 2010; Zhihua Zhang, James T. Kwok, and Dit-Yan Yeung, “Parametric distance metric learning with label information,” in Proc. Int'l Joint Conf. on Artificial Intelligence, 2003, pp. 1450-1452; Kai Zhang, Ming Tang, and James T. Kwok, “Applying neighborhood consistency for fast clustering and kernel density estimation,” in Proc. IEEE Conf. on Computer Vision and Pattern Recognition, 2005, pp. 1001-1007) attempt to learn metrics for the kNN classifier by finding feature weights adapted to individual test samples in a database.

Some of the early work in metric learning for medical image registration includes that of Leventon et al. (Michael E. Leventon, W. Eric, and W. Eric L. Grimson, “Multi-modal volume registration using joint intensity distributions,” in Int'l Conf. on Medical Image Computing and Computer Assisted Intervention. 1998, pp. 1057-1066, Springer) and Sabuncu et al (Mert R. Sabuncu and Peter Ramadge, “Using spanning graphs for efficient image registration,” IEEE Transactions on Image Processing, vol. 17, 2008). These methods are based on learning an underlying joint distribution from a training set. A new registration is then evaluated by computing its joint distribution and optimizing a cost function, (such as a divergence function) with the learned data. The above mentioned methods are all based on generative models. More recently, discriminative techniques have also been applied for learning similarity metrics within certain imaging domains. Zhou et al. (Shaohua Kevin Zhou, Bogdan Georgescu, Dorin Comaniciu, and Jie Shao, “Boostmotion: Boosting a discriminative similarity function for motion estimation,” in Proc. IEEE Conf. Computer Vision and Pattern Recognition. 2006, pp. 1761-1768, IEEE Computer Society) apply Logitboost to learn matches for motion estimation in echocardiography. Muenzing et al. (Sascha E. A. Muenzing, Keelin Murphy, Brain van Ginneken, and Josien P. W. Pluim, “Automatic detection of registration errors for quality assessment in medical image registration,” in Proc. SPIE Conf. on Medical Imaging, 2009, vol. 7259, p. 72590K) apply SVMs to learn matches for registration of lung CT. Seshamani et al (S. Seshamani, R. Kumar, P. Rajan, S. Bejakovic, G. Mullin, T. Dassopoulos, and G. Hager, “Detecting registration failure,” in Proc. IEEE international Symposium of Biomedical Imaging, 2009, pp. 726-729) apply Adaboost to learn matches in capsule endoscopy. All these methods are supervised and are used in conjunction with one registration method to simply eliminate matches that are incorrect.

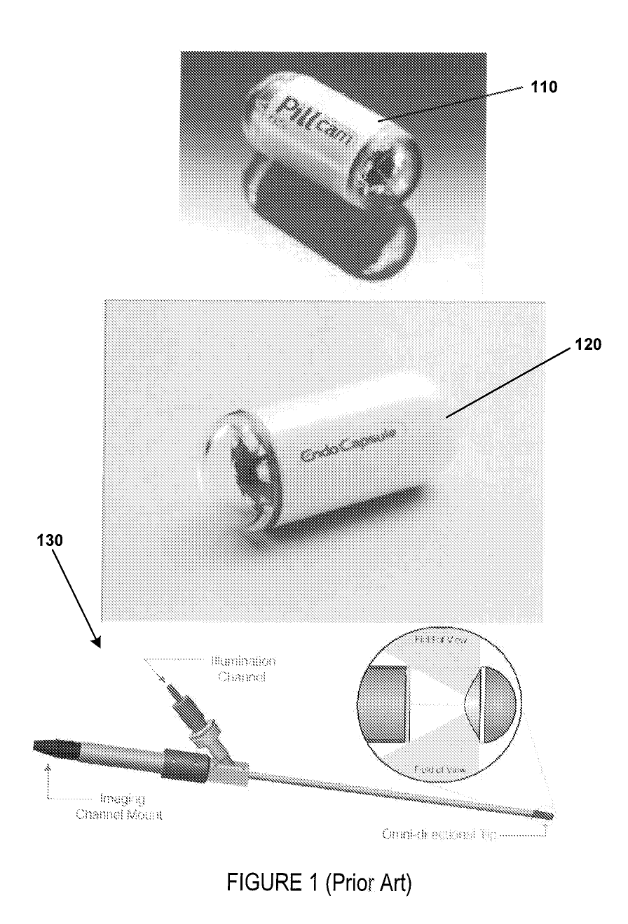

One embodiment of the invention matches lesions in CE images. Automated matching of regions of interest may reduce evaluation time. An automated matching system may allow for the clinician to select a region of interest in one image and use this to find other instances of the same region to present back to the clinician for evaluation. Crohns disease, for example, may affect any part of the gastrointestinal tract and may be characterized by discrete, well-circumscribed (punched-out) erosions and ulcers 910 of FIG. 9. However, since the capsule imager FIG. 1, 110 and 120 is not controllable, there may be a large variation in the appearance of CD lesions in terms of illumination, scale and orientation. In addition, there may also be a large amount of background variation present in the GI tract imagery. Metamatching may be used to improve match retrieval for this type of data.

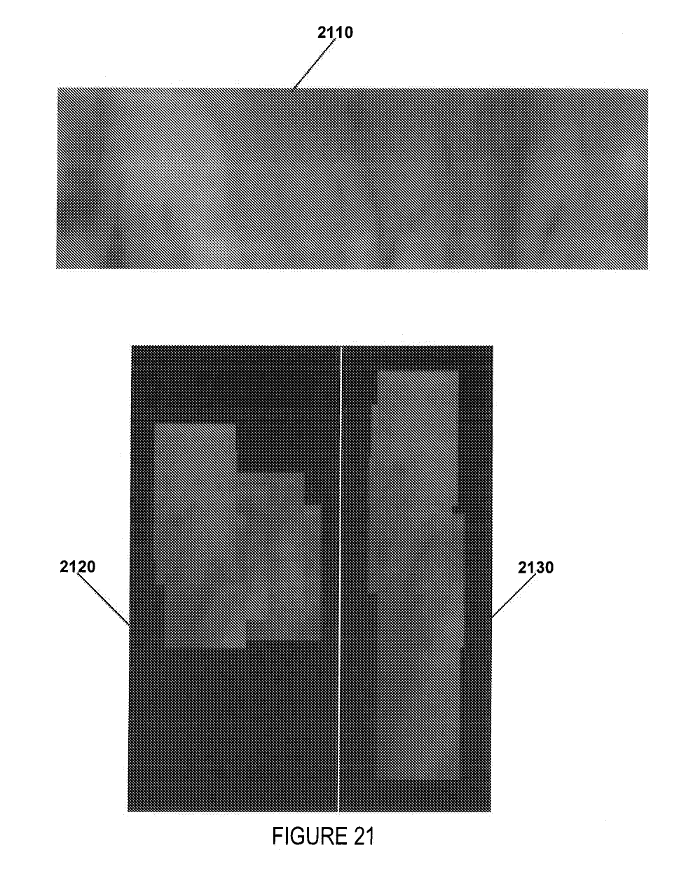

As opposed to CE, contact hysteroscopy enables the early diagnosis of uterine cancer to be performed as an in-office procedure. A contact hysteroscope 130 of FIG. 1 consists of a rigid shaft with a probe at its tip, which may be introduced via the cervix to the fundus of the uterus. The probe may feature a catadioptric tip that allows visualization of 360 degrees of the endometrium perpendicular to the optical axis. The detail on the endometrial wall captured by this device may be significantly higher compared to traditional hysteroscopic methods and may allow for cancerous lesions to be detected at an earlier stage. However, the field of view captured by any single image frame may be only about 2 mm. 2110 of FIG. 21 shows an example raw image from a contact hysteroscope.

Mosaicking consecutive video frames captured from a hysteroscopic video sequence may provide improved visualization for the clinician. Video mosaicking may generate an environment map from a sequence of consecutive images acquired from a video. The procedure may involve registering images, followed by resampling the images to a common coordinate system so that they may be combined into a single image. For contact hysteroscopic mosaicking, one embodiment uses direct registration of images (S. Seshamani, W. Lau, and G. Hager, “Real-time endoscopic mosaicking,” in Int'l Conf. on Medical Image Computing and Computer Assisted Intervention, 2006, vol. 9, pp. 355-363; S. Seshamani, M. D. Smith, J. J. Corso, M. O. Filipovich, A. Natarajan, and G. D. Hager, “Direct Global Adjustment Methods for Endoscopic Mosaicking,” in Proc. SPIE Conf. on Medical Imaging, 2009, p. 72611D) with large areas of overlap (e.g., for example, more than 80 percent overlap between images being registered). This procedure may rely on an initial gross registration estimate (to, for example, the closest pixel), followed by subpixel optimization. Although the motion may be small between consecutive frames, it is not necessarily consistent since the endoscopic imager may be controlled manually. FIG. 21, 2120 and 2130 show two examples of endometrial mosaics generated with frame-to-frame estimates of corresponding regions. It can be noted that due to the lack of features in these images, there are several incorrect estimates which may affect the overall visualization. Metamatching may be used to generate a set of match estimates and may decide which one (if any) is suitable for the visualization.

### Overview of Metamatching

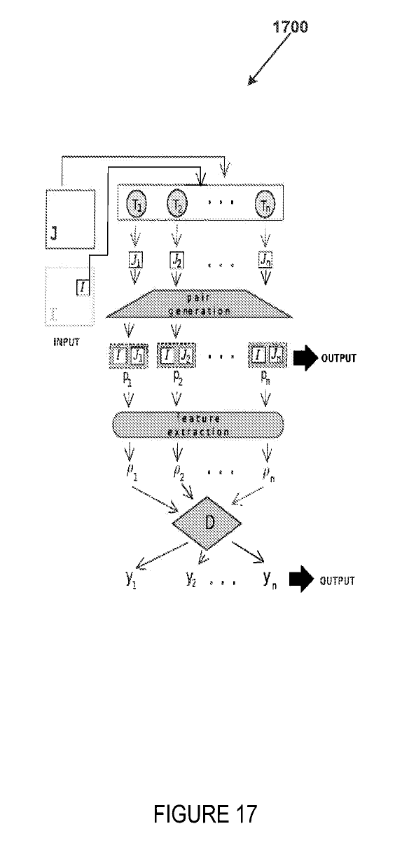

FIG. 17 depicts an overview of a metamatching procedure 1700. In 1700, the input to the algorithm include a region I and image J. (T₁ . . . Tₙ) are the set of matchers which compute an estimate of a region corresponding to I in J. These estimates J₁ . . . Jₙ are then combined with I to generate match pairs p₁ . . . pₙ. These pairs are then represented with feature vectors ρ₁ . . . ρₙ and finally input to a decision function D which estimates the labels y₁ . . . yₙ that corresponds to each pair.

The objective of metamatching may be as follows: Given a region I and image J, find a region within J which corresponds to region I. An example metamatching system is shown in 2100 of FIG. 21, which uses a set of matchers and a decision function to perform this task. Metamatcher may be defined as: [symbol] ={ [symbol] , D} where [symbol] may be a set of n matchers: [symbol] ={T₁, . . . Tₙ} and D may be a decision function. Given I and J, each matcher Tᵢ∈ [symbol] estimates a region which corresponds to I: Tᵢ(I, J) [symbol] J^{T}^{ᵢ}^{J} Every J^{T}^{ᵢ}^{J} together with I forms a match pair (I, J^{T}^{ᵢ}^{J}) thus generating a set: P={pᵢ|pᵢ=(I, J^{T}^{ᵢ}^{J}), i=1 . . . n}. A representation function ƒ is then applied to each pair to generate a feature vector ρ for each pair: ρᵢ=ƒ(pᵢ).

The decision function D may then use these pair representations to estimate which of these match pairs are correct matches. If none of the match pairs are qualified as correct, the metamatching algorithm may determine that there is no match present for region I in image J. If one is correct, the algorithm may conclude that a correct match has been found. If more than one match pair may be qualified as correct, one of the matches may be chosen. In one embodiment of the invention, we use SVM based ordinal regression to rank matches and select the best match. However, in most cases, a selection algorithm may not be required since matches which have been retrieved by Tᵢ's and qualified as correct by D are likely to be the same result. One embodiment of this invention is focused on the problem of optimizing the performance of the decision function D with respect to the matchers. This performance may be defined as the harmonic mean of the system which evaluates the system in terms of both recall and precision.

### Decision Function Design

An element of metamatching may be the use of a decision function. In one embodiment, given a pair of regions p=(I; J), a decision function D may be designed which can determine whether these two regions correspond or not. More formally, D may be a binary classification function whose input is p and the desired output may be a variable y which represents membership of pair p to the class of corresponding regions which may be denoted C₁ or the class of non-corresponding regions which may be denoted C₂. One embodiment selects y=1 to correspond to class C₁ and y=−1 to correspond to class C₂. The task of D may be to predict the output y given p:

$$y = D ( p ) = D ( I , J ) = \{ \begin{matrix}1 , & if p ∈ {C}_{1} \\ - 1 & if p ∈ {C}_{2}\end{matrix}$$

In one embodiment, given a set of pairs and their associated labels, D may be trained using supervised learning techniques to perform this binary classification task.

1) Training the Decision Function: Given a set of r pair instances and their associated labels,

[symbol]

ₜᵣₐᵢₙ={(p_{q},y_{q})|y_{q}∈{1,−1}, q=1 . . . r}

In one embodiment each pair may be represented as an m vector using some representation function ƒ: ρ=ƒ(p), ƒ∈ [symbol] ^{m} This may generate a training set:

Πₜᵣₐᵢₙ={(ρ_{q},y_{q})|ρ=ƒ(p),(p_{q},y_{q})∈Lₜᵣₐᵢₙ, q=1 . . . r}

In this embodiment, D may be trained using any standard classifier to perform this binary classification. To account for order invariance, D may be pairwise symmetric, ie: D(I,J)=D(J,I). There may be two ways of ensuring this property, for example, using a pairwise symmetric representation, (e.g., for example, ƒ(I, J)=ƒ(J, I)) or using a pairwise symmetric classification function.

### Selection of Matchers

In one embodiment of the invention, the performance of metamatching systems may be evaluated and compared to determine a set of matchers that may be used in conjunction with a decision function to obtain the best performance. A common measure used to determine the performance of a system (taking both the precision as well as recall into consideration) may be the harmonic mean or F measure (C. J. van Rijsbergen and Ph. D, Information Retrieval, Butterworth, 1979). This value may be computed as follows:

$$F = \frac{2 P * R}{P + R}$$

where P may be the precision of the system and the R may be the recall rate of the system. A higher F measure therefore may indicate better system performance. In one embodiment, a metamatching system may include one matcher and a decision function: [symbol] ^{l}={T₁, D} This system may be presented a set of r ROI-image sets:

{(I_{q},J_{q}), q=1 . . . r}

One embodiment of the invention generates matches and identifies the correct ones. The metamatcher may applies T₁ to each of the r ROI-image sets. For each ROI-image set (I_{q},J_{q}), T₁ may locate one prospective matching region J_{q}^{T}^{₁}^{J}. This matching region together with the ROI (from the ROI-image set) may form an ROI pair: (I_{q}, J_{q}^{T}^{₁}^{J}), which may generate a total of r ROI pairs.

Each ROI pair (I_{q}, J_{q}^{T}^{₁}^{J}) may be assigned a ground truth label y_{q}*, y_{q}*=1 when J_{q}^{T}^{₁}^{J} is similar to I_{q} and −1 otherwise. The trained decision function D may then compute a label y_{q} for each ROI pair. A label of y_{q}=1 may indicate that the pair may be qualified as similar by the decision function and y_{q}=−1 may indicate that the pair may be qualified as dissimilar by the decision function.

Thus, given the ground truth labels y_{q}* and the estimated labels y_{q}, we may obtain four types of ROI pairs: true positives, false positives, true negatives and false negatives. Table VI shows an example four types of ROI pairs:

TABLE VI

| Type | Meaning |
|---|---|
| True Positive | y* = 1 and y = 1 |
| False Positive | y* = −1 and y = 1 |
| True Negative | y* = −1 and y = −1 |
| False Negative | y* = 1 and y = −1 |

The number of ROI pairs that fall into each category may be computed empirically. Each of these numbers may be defined as: TP_{T}_{₁}=Number of true positives generated by T₁ and D, FP_{T}_{₁}=Number of False Positives generated by T₁ and D, TN_{T}_{₁}=Number of True Negatives generated by T₁ and D, FN_{T}_{₁}=Number of False Negatives generated by T₁ and D. The precision of the system may be computed as:

$$P = \frac{{TP}_{{T}_{1}}}{{TP}_{{T}_{1}} + {FP}_{{T}_{1}}}$$

In one embodiment, the system may be a matcher and classifier combination and the recall of the system may be defined as follows:

$$R = \frac{{TP}_{{T}_{1}}}{{TP}_{{T}_{1}} + {FP}_{{T}_{1}} + {TN}_{{T}_{1}} + {FN}_{{T}_{1}}} = \frac{{TP}_{{T}_{1}}}{r}$$

The total number of positives may be defined as:

POS_{T}_{₁}=TP_{T}_{₁}+FP_{T}_{₁}

The F measure may be written as:

$$F = \frac{2 {TP}_{{T}_{1}}}{1 + {POS}_{{T}_{1}}}$$

A metamatcher made up of n matchers and a decision function may be defined as:

[symbol]

ⁿ={{T₁ . . . Tₙ}D}

By definition, the metamatcher [symbol] ⁿ may locate a correct match if any one of its matchers Tᵢ locates a correct match. The number of true positives generated by this metamatcher may be computed as:

$${TP}_{{ϒ}^{n}} = ( {TP}_{{T}_{1}} ⋁ {TP}_{{T}_{2}} ⋁ … ⋁ {TP}_{{T}_{n}} ) = {\sum }_{i = 1}^{n} {TP}_{{T}_{i}} - {\sum }_{i < i = 1}^{n} ( {TP}_{{T}_{i}} ⋀ {TP}_{{T}_{j}} ) - … {\sum }_{i < j … n = 1}^{n} ( {TP}_{{T}_{i}} ⋀ {TP}_{{T}_{j}} ⋀ … ⋀ {TP}_{{T}_{n}} )$$

where (TP_{T}_{ᵢ} [symbol] TP_{T}_{ⱼ}) may be the number of True Positives that are generated from matcher Tᵢ and matcher Tⱼ (the intersection) with D. Similarly, one may compute the total number of positives as:

$${POS}_{{ϒ}^{n}} = ( {POS}_{{T}_{1}} ⋁ {POS}_{{T}_{2}} ⋁ … ⋁ {POS}_{{T}_{n}} ) = {\sum }_{i = 1}^{n} {POS}_{{T}_{i}} - {\sum }_{i < j = 1}^{n} ( {POS}_{{T}_{i}} ⋀ {POS}_{{T}_{j}} ) - … {\sum }_{i < j … n = 1}^{n} ( {POS}_{{T}_{i}} ⋀ {POS}_{{T}_{j}} ⋀ … ⋀ {POS}_{{T}_{n}} )$$

where (POS_{T}_{ᵢ} [symbol] POS_{T}_{ⱼ}) may be the number of Positives qualified by D for the matches generated by matcher Tᵢ and matcher Tⱼ (the intersection). The harmonic mean of this metamatcher [symbol] ⁿ may be computed as:

$${F}_{{ϒ}^{n}} = \frac{2 {TP}_{{ϒ}^{n}}}{1 + {POS}_{{ϒ}^{n}}}$$

### Selecting an Optimal Set of Matchers

In an embodiment of the invention, the addition of a new matcher may not always increase the performance of the overall precision-recall system. This may be observed in the equation directly above, where the number of true positives (TP) is not increased but the number of positives classified by the decision function (POS) does increase with the addition of a new matcher. This depends on how well the decision function can classify matches generated by the new matcher. For n prospective matchers, there may exist 2ⁿ−1 possible types of metamatchers that can be generated (with all combinations of matchers). This number grows exponentially with the number of matchers under consideration.

### Representation Functions for a Match Pair

In one embodiment, given a match pair p=(I, J), the representation function ƒ may generate w scalar or vector subcomponents d₁ . . . d_{w}. These subcomponents may then be stacked up to populate a feature vector ρ as follows:

$$f ( p ) = ρ = [ \begin{matrix}{d}_{1} \\ ⋮ \\ {d}_{w}\end{matrix} ]$$

Each dⱼ may contain similarity information between the two images. For each d, there may be two choices to be made. First, a choice of a region descriptor function Rⱼ. Second, a choice of a similarity measure s between region descriptors of I and J: dⱼ=sⱼ(Rⱼ(I), Rⱼ(J)). For an embodiment to satisfy the pairwise symmetric property described earlier, the similarity measure may also satisfy: sⱼ(Rⱼ(I), Rⱼ(J))=dⱼ=sⱼ(Rⱼ(J), Rⱼ(I))

### Selection of a Region Descriptor:

Almost all region descriptors are either structural or statistical (Sami Brandt, Jorma Laaksonen, and Erkki Oja, “Statistical shape features in content-based image retrieval,” in Proc. IEEE Int'l Conf on Pattern Recognition, 2000, pp. 6062-6066) in nature, and some can be combinations of both. In one embodiment of the invention the following features may be applied:

### Structural

Image Intensities: This descriptor may consist of a vector containing the intensity values at all locations in the image. For this descriptor to be used, two regions may be resampled to the same size in order to be comparable.
Patch Based Mean Pixel: Here, the image may be broken down into a fixed number of blocks and the mean intensity value may be computed for each block. For example, 16 blocks may be used and the image representation may be a 16-vector.
Condition Numbers: The vector of spatial gradients containing values for each pixel Iₓ and I_{y} are first computed and stacked to generate an NX2 Jacobian matrix Iⱼ. The condition number of this Jacobian represents a measure of how well structured (in terms of gradients) the region is (C. Harris and M. Stephens, “A combined corner and edge detector,” in Proc. Fourth Alvey Vision Conference, 1988, pp. 147-151).
Homogeneous Texture Descriptor (MPEG 7): This descriptor may characterize properties of texture in the region based on the assumption that texture may be homogeneous in the region. The descriptor is a 62-vector resulting from features extracted from a bank of orientation and scale-tuned Gabor filters (BS Manjunath, JR Ohm, VV Vasudevan, and A Yamada, “Color and texture descriptors,” IEEE Transactions on circuits and systems for videotechnology, vol. 11, no, 6, pp. 703-715, 2001).
Gist features: This descriptor may represent the dominant spatial structure of the region, and may be based on a low dimensional representation called the spatial envelope (Aude Oliva and Antonio Torralba, “Modeling the shape of the scene: A holistic representation of the spatial envelope,” International Journal of Computer Vision, vol. 42, pp. 145-175, 2001).

### Statistical

Histograms: A histogram may be a representation of the distribution of intensities or colors in an image, derived by counting the number of pixels of each of given set of intensity or color ranges in a 2D or 3D space.
Invariant Moments: These may measure a set of image statistics that are rotationally invariant. They may include: mean, standard deviation, smoothness, third moment, uniformity and entropy. In one embodiment of the invention the implementation used of this descriptor is from (Rafael C. Gonzalez, Richard E. Woods, and Steven L. Eddins, Digital Image Processing Using MATLAB, Gatesmark Publishing, 1st edition, 2004).
Haralick features: These may be a set of metrics of the co-occurence matrix for an image, which may measure the textural features of the image (R. M. Haralick, Shanmugan K., and I. Dinstein, “Textural features for image classification,” IEEE Transactions on Systems, Man, and Cybernetics, vol. 3, no. 6, pp. 610-621, 1973).

### Combined

Spatially Weighted Histograms: This may be a histogram where pixels may be weighted by their location. In one embodiment of the invention, pixels closer to the center are weighed with a higher weight than pixels at the outer edge of the region.
   In one embodiment, except for the histogram and weighted histogram measures, all other measures may be specified for gray scale images. The color version may be computed by applying the feature to each channel of the color image.

### Similarity Measures

### Scalar Functions

A distance metric is a scalar value that represents the amount of disparity between two vectorial data points. Distance metrics are pairwise symmetric by definition and may be used to populate a feature vector that may represent similarity between images in the pair. The low dimensionality provided by this

representation is one of its main advantages. However, in some cases, the loss of information due to dimensional reduction may be a drawback for the type of classification as applied in one embodiment of the invention. The range of such metrics may fall into one of three categories:
Accuracy based metrics: These measures may compute a specific cost function between the two images. The measures may be those that are used for optimization for computation of a registration. (e.g.: SSD error, mutual information, etc).
Stability based metrics: These may measure how stable the match is by computing local solutions. Examples of such measures may include patch based measures. (These may include metrics and statistics computed between patch based region descriptors).
Consistency based metrics: These metrics may compute how consistently the registration transformation computed the match. The forward backward check (Heiko Hirschmller and Daniel Scharstein, “Evaluation of cost functions for stereo matching.,” in CVPR. 2007, IEEE Computer Society) used in stereo matching is an example of this.
   Each type of region descriptor described in the previous section may have an appropriate set of meaningful metrics. The region descriptors along with their associated metrics are summarized in Table VII. The feature vector generated, for example, by using all of the region descriptors and metrics shown in the table would be of length 9. For each type of dataset (e.g., but not limited to, hysteroscopy and capsule endoscopy), descriptor selection may be carried out by computing ROC curves for using each metric separately as a classifier.

TABLE VII

| | |
|---|---|
| Region Descriptor (vector) | Metric (scalar) |
| Image Intensities | SSD (Euclidean) |
| Region Condition Numbers | Ratio (smaller/larger) |
| Homogeneous Texture Descriptors | HTD Shuffle Distance [31] |
| GIST features | Euclidean |
| Patch Intensities (Grayscale and 3 color | Euclidean |
| bands) | |
| Histograms | Bhattacharya Distance |
| Haralick Descriptors | Canberra Distance |
| Image Moments | Euclidean Distance |
| Spatially Weighted Histograms | Bhattacharya Distance |

### Vector Functions

In another embodiment, the similarity representations may be generated by computing element wise squared difference of the values within each region descriptor as follows:

dⱼ=sⱼ(Rⱼ((I)),Rⱼ(J))=(Rⱼ(I)−Rⱼ(J))²

Each of the dⱼ's representations may be the same length as the region descriptors. One advantage of using this type of feature descriptor may be the reduction of information loss. However, a drawback may be that the use of large region descriptors and the increase in numbers of region descriptors may cause the feature vectors generated to be of a very high dimension.

### Classification Methods for the Decision Function

In one embodiment with a set of matched pairs represented as feature vectors, a classifier is computed that may distinguish correct matches from incorrect ones. The following standard classifiers may be used: Nearest Neighbors (Christopher M. Bishop, Pattern Recognition and Machine Learning (Information Science and Statistics), Springer-Verlag New York, Inc., Secaucus, N.J., USA, 2006), Support Vector Machines (Bernhard Scholkopf, Christopher J. C. Burges, and Alexander J. Smola, Eds., Advances in kernel methods: support vector learning, MIT Press, Cambridge, Mass., USA, 1999; Vladimir N. Vapnik, The nature of statistical learning theory, Springer-Verlag New York, Inc., New York, N.Y., USA, 1995), Linear Discriminant Analysis and Boosting (P. Viola and M. Jones, “Robust real-time face detection,” International Journal of Computer Vision, vol. 57, no. 2, pp. 137-154, 2004).

### Generating Training and Testing Pairs for Capsule Data

In an exemplary embodiment of the invention using capsule endoscopy, the dataset may consist of sets of images containing the same region of interest. In one embodiment, centers of corresponding regions of interest are manually annotated. The set of N images in which the same region appears is defined as: [symbol] ={I₁, I₂ . . . I_{N}} and the set of all the annotated regions as [symbol] ₀={I₁, I₂ . . . I_{N}} where Iₖ is the region extracted from the kth image Iₖ in the set. Note that this index k (which refers to the image index) may be different from the index i used above to denote the index of the matcher.

Every pair of ROIs (Iₖ, Iₗ) in S₀ may form a match pair. However, this may not be used as a training set since it may not contain any negative examples. Instead, matchers may be used to generate examples of positive and negative match pairs.

For example, given a matcher T, a region Iₖ and an image Iₗ we may compute an estimate of a corresponding region: T(lₗ, Iₗ) [symbol] Iₗ^{T}^{ₗ}^{I}^{ₖ} to generate a pair (Iₖ, Iₗ^{T,I}^{ₖ}). For a given matcher, such pairs may be computed between every region in the set S₀ and every image in [symbol] .

Labels may be generated for the pairs as follows. The Euclidean distance between the center of Iₗ^{T,I}^{ₖ} and Iₗ may be defined as distₖₗ. The associated label y(Iₖ, Iₗ^{T,I}^{ₖ}) for the pair (Iₖ, Iₗ^{T,I}^{ₖ}) may be generated as:

$${y}_{( {I}_{k} , {I}_{l}^{T , {I}_{k}} )} = \{ \begin{matrix}1 , & if {dist}_{kl} < γ \\ - 1 & otherwise\end{matrix}$$

where γ>0 may be a threshold selected for each training model. The match data set generated by these N images in which the same region appears may contain labeled pairs:

$${capsule}_{} = \{ ( ( {I}_{k} , {I}_{l}^{T , {I}_{k}} ) , {y}_{( {I}_{k} , {I}_{l}^{T , {I}_{k}} ) )} \vert k , l - 1 … N , k \neq l \}$$

Match datasets may be generated for all such sets of images and combine them to form the full dataset. This full dataset may be used for training and testing. Cross validation may be performed to partition the data into independent training and testing sets.

### Generating Training and Testing Pairs for Endometrial Data

In an embodiment where endometrial imaging is used, data may consist of a video sequence where consecutive images may be registered at a finer level. Hence, training data may be obtained by generating positive and negative examples by offsetting matching regions. This data may be referred to as N-offset data. N-offset data may be generated by sampling regions at various offsets from a manually annotated center. Given [symbol] and S₀ as described in the previous section, we define a displaced region Iₗ^{c} s a region in Iₗ that may be at a displacement of c pixels from the manually annotated region Iₗ. The set of all regions at a particular displacement value c may be denoted as S_{c}.

A training pair may be generated as (Iₖ⁰, Iₗ^{c}) (a training pair may include an region from S₀). The set of all training pairs generated by the set of images in which the same region appears may be written as: P_{Endometrical}={(Iₖ⁰, Iₗ^{c})|k, l=1:N} and may include two types of pairs in equal numbers: (Iₖ⁰, Iₗ^{c}) where c<γ and (Iₖ⁰, Iₗ^{c}) where c>γ. This may assure both positive and negative examples in the training set. The associated classifications for pairs may be computed as in the previous section to generate the set of labelled data:

[symbol]

_{Endometrial}={((Iₖ⁰,Iₗ^{c}),y₍ₗ_{ₖ}_{⁰}_{,l}_{ₗ}_{^{c}}₎)|(Iₖ⁰,Iₗ^{c})∈P_{Endometrial}}

In one embodiment, this is generated using all sets of images in which the same region occurs and may combine them to form the fill training set. The testing set may be generated using matchers, using the methodology described above to generate [symbol] _{capsule},

### Metamatching for Lesion Finding in Capsule Endoscopy

In one embodiment, lesions were selected and a search for the corresponding region was performed on all other images in the lesion set using the following four matchers: NCC template matching (Matcher 1), SIFT (Matcher 2), weighted histogram matching (Matcher 3) and color weighted histogram matching (Matcher 4). Each pair was then represented using the scalar (metric) representation functions and the vector (distance squared) representation functions described above using the following region descriptors: Homogeneous Texture, Haralick features, Spatially weighted histograms, RGB histograms, Moments, Normalized mean patch intensities, Normalized patch condition numbers, Local Binary Patterns, GIST and Sum of Squared Differences of Intensities (SSD).

### Augmenting Capsule Endoscopy Diagnosis: A Similarity Learning Approach

In one embodiment of the invention, the invention improves on the diagnostic procedure of reviewing endoscopic images through two methods. First, diagnostic measures may be improved through automatic matching for locating multiple views of a selected pathology. Seshamani et al. propose a meta matching procedure that incorporates several simple matchers and a binary decision function that determines whether a pair of images are similar or not (Seshamani, S., Rajan, P., Kumar, R., Girgis, H., Mullin, G., Dassopoulos, T., Hager, G.: A meta registration framework for lesion matching. In: MICCAI. (2009) 582-589). The second diagnostic improvement may be the enhancement of CD lesion scoring consistency with the use of a predictor which can determine the severity of the lesion based on previously seen examples. Both of these problems may be approached from a similarity learning perspective. Learning the decision function for meta matching may be a similarity learning problem (Chen, Y., Garcia, E. K., Gupta, M. R., Rahimi, A., Cazzanti, L.: Similarity-based classification: Concepts and algorithms. JMLR 10 (March 2009) 747-776)). Lesion severity prediction may be a multi-class classification problem which involves learning semantic classes of lesions based on appearance characteristics. Multi-class classification may also be approached from a similarity learning approach as shown in (Chen, Y., Garcia, E. K., Gupta, M. R., Rahimi, A., Cazzanti, L.: Similarity-based classification: Concepts and algorithms. JMLR. 10 (March 2009) 747-776; Cazzanti, L., Gupta, M. R.: Local similarity discriminant analysis. In: ICML. (2007)). In one embodiment of the invention, both problems are approached as supervised pairwise similarity learning problems (Vert, J. P., Qiu, J., Noble, W. S.: A new pairwise kernel for biological network inference with support vector machines. BMC Bioinformatics 8(S-10) (2007); Kashima, H., Oyama, S., Yamanishi, Y., Tsuda, K.: On pairwise kernels: An efficient alternative and generalization analysis. In: PAKDD. (2009) 1030-1037; Oyama, S., Manning, C. D.: Using feature conjunctions across examples for learning pairwise classifiers In: ECML. (2004)).

### Pairwise Similarity Learning

The pairwise similarity learning problem may be considered as the following: given a pair of data points, determine if these two points are similar, based on previously seen examples of similar and dissimilar points. A function that performs this task may be called a pairwise similarity learner (PSL). A PSL is may be made up of two parts: a representation function, and a classification. In addition, the PSL may also be required to be invariant to the ordering of pairs. One method of assuring order invariance is by imposing a symmetry constraint on the representation function (Seshamani, S., Rajan, P., Kumar, R., Girgis, H., Mullin, G., Dassopoulos, T., Hager, G.: A meta registration framework for lesion matching. In: MICCAI. (2009) 582-589). However, doing so may introduce a loss of dimensionality and possibly a loss of information that may be relevant for the classification task. Order invariance of the PSL may also be ensured by imposing symmetry constraints on the classifier. Such a classification function may be referred to as a pairwise symmetric classifier. Several SVM-based pairwise symmetric classifiers have been proposed. Within the SVM framework, symmetry may be imposed by ensuring that the kernel function satisfies order invariance. In prior work concerning pairwise symmetric classifiers, a pair may be described by only one type of feature and the underlying assumption is that one distance metric holds for the entire set of points. However, this assumption may not hold when multiple features are used to describe data. The area of Multiple Kernel Learning (Rakotomamonjy, A., Bach, F. R., Canu, S., Grandvalet, Y.: Simplemkl. JMLR 9 (2008); Varma, M., Babu, B. R.: More generality in e_cient multiple kernel learning. In: ICML. (June 2009) 1065-1072; (Gehler, P., Nowozin, S.: Let the kernel figure it out: Principled learning of preprocessing for kernel classifiers. In: CVPR. (2009)) has investigated several methods for combining features within the SVM framework. In one embodiment, the invention uses a novel pairwise similarity classifier for PSL using nonsymmetric representations with multiple features.

### Mathematical Formulation

One embodiment may include a pair of images (I,J) and a set X consisting of m image descriptors (features). Applying any Xᵢ∈X to each image in the pair may generate a representation x̅=(x₁, x₂) where x₁{Xᵢ(I)} and x₂={Xᵢ(J)}. A label y∈{1, −1} may be associated with x̅, where y=1 may imply a pair of similar images and y=−1 may imply a pair of dissimilar images. The PSL problem may be written as follows: given a training set with n image pair representations and their associated labels [symbol] ₘ={( x̅ᵢm yᵢ)|i=1 . . . n}, compute a classifier C that may predict the label of an unseen pair x̅:

$$C ( \tilde{x} ) = C ( ( {x}_{1} , {x}_{2} ) ) = \{ \begin{matrix}1 , & if \tilde{x} represents a pair of similar images \\ - 1 & otherwise\end{matrix}$$

Order invariance may require C((x₁, x₂))=C((x₂, x₁)). We refer to this as the pairwise symmetric constraint. An SVM trained on the set T may classify an unseen pair x̅=(x₁, x₂) as:

$$C ( \tilde{x} ) = {\sum }_{∀ ( {x}_{1} , {y}_{1} ) ∈ T} {α}_{i} {y}_{i} K ( \tilde{x} , {\tilde{x}}_{i} ) + b$$

where b and αᵢ's may be learned from training examples and K is a Mercer kernel. This classifier may satisfy the pairwise symmetric constraint if K satisfies: K( x̅, x̅ᵢ)=K((x₁, x₂), (xᵢ₁, xᵢ₂))=K((x₂, x₁), (xᵢ₁, xᵢ₂)). Such a kernel may be referred to as a pairwise symmetric kernel (PSK).

### PSKs for One Descriptor

Mercer Kernels may be generated from other Mercer Kernels by linear combinations (with positive weights) or element wise multiplication (Cristianini, N., Shawe-Taylor, J.: An Introduction to Support Vector Machines: and Other Kernel-Based Learning Methods. Cambridge University Press (2000)). This idea may be used to generate PSKs from simpler Mercer Kernels. Assuming that we have two pairs: (x₁, x₂) and (x₃, x₄) and a base mercer kernel K, which may operate on a pair of points. A PSK (which may operate on two pairs of points) may be computed by symmetrization of the base kernel. Other work has shown that a second order PSK called the MLPK may be introduced (Vert, J. P., Qiu, J., Noble, W. S.: A new pairwise kernel for biological network inference with support vector machines. BMC Bioinformatics 8(S-10) (2007)): \hat{K}((x₁, x₂), (x₃, x₄))=(K(x₁, x₃)+K(x₂, x₄)−K(x₁, x₄)−K(x₂, X₃))². This kernel may be a linear combination of all second order combinations of the four base Mercer kernels. This kernel may be rewritten in terms of 3 PSKs as \hat{K}=K₁+2K₂−2K₃ where:

K₁=K(x₁,x₃)²+K(x₂,x₄)²+K(x₁,x₄)²+K(x₂,x₃)²

K₂=K(x₁,x₃)K(x₂,x₄)+K(x₁,x₄)K(x₂,x₃)

K₃=K(x₁,x₃)K(x₁,x₄)+K(x₁,x₃)K(x₂,x₃)K(x₂,x₄)K(x₁,x₄)+K(x₂,x₄)K(x₂,x₃)

The MLPK kernel may be different from a second order polynomial kernel due to the additional base kernels it uses. A classifier trained with the MLPK kernel may be comparable to a classifier trained with a second order polynomial kernel on double the amount of data (with pair orders reversed). SVM complexity may be exponential in the number of training points (in the worst case) (Gärtner, B., Giesen, J., Jaggi, M.: An exponential lower bound on the complexity of regularization paths. CoRR (2009)). Secondly, a larger training dataset may generate more support vectors which increase run time complexity (classification time). Thus, the PSK may be greatly beneficial in the reduction of both training and classification time.

PSKs with More than One Descriptor
In one embodiment, with one descriptor, 3 second order PSKs (K₁, K₂ and K₃) may be obtained. So, given a set of m descriptors, we may generate a total of 3m second order PSKs: Q={Kᵢ′|i=1 . . . 3m}. The problem now becomes the following: Given a set of PSKs find a weight vector d∈ [symbol] ^{3m} that can generate a kernel \hat{K}=Σᵢ^{3m}dᵢKᵢ′ where dᵢ∈d, Kᵢ′∈Q. In one embodiment, Simple Multiple Kernel Learning (SimpleMKL) may be used for automatically learning these weights (Rakotomamonjy, A., Bach, F. R., Canu, S., Grandvalet, Y.; Simplemkl. JMLR 9 (2008)). This method may initialize the weight vector uniformly and may then perform a gradient descent on the SVM cost function to find an optimal weighting solution. A Generalized Pairwise Symmetric Learning (GPSL) training algorithm, used in one embodiment, is outlined below.
Input: Training set [symbol] ₘ and m base kernels.
Output: Weight Vector d_{best}, SVM parameters α and b
For each of the m features, compute K₁, K₂ and K₃ (as described above) between all training pairs to generate the set Qₜᵣₐᵢₙ{Kᵢ′|i=1 . . . 3m}
Apply SimpleMKL to find a weight vector d_{best},
Learn the SVM parameters α and b using a kernel generated as a linear combination of kernels in Q using d_{best}.
   To predict similarity of an unseen pair x̅:
Compute the set Qₜₑₛₜ using the test point and training examples.
Generate a linear combination of these kernels using d_{best}.
Predict the similarity of the pair using the learned α and b.

### Multiclass Classification

The multiclass classification problem for images may be as follows: given a training set consisting of k images and their semantic labels [symbol] ={(Iᵢ, lᵢ)|i=1 . . . k, lᵢ∈{1 . . . p}}, where Iᵢs are the images and lᵢs are the labels belonging to one of p classes, compute a classifier that may predict the label of an unseen image I. From a similarity learning approach, this problem may be reformulated as a binary classification and voting problem: given a training set of similar and dissimilar images, compute the semantic label of a new unseen image I. This may require two steps: 1) Learning similarities, and 2) Voting, to determine the label of an unseen image. One embodiment may use the same method outlined in the GPSL algorithm above for similarity learning. Voting may then be performed by selection of n voters from each semantic class who decide whether or not the new image is similar or dissimilar to themselves. We refer to this algorithm as GPSL-Vote:
Given [symbol] , compute a new training set consisting of all combinations of pairs and their similarity labels: [symbol] ={((Iᵢ, Iⱼ)ₖ, yₖ)|(Iᵢ, lᵢ), (Iⱼ, lⱼ)∈ [symbol] , yₖ∈{1, −1}} where yₖ=1 if lᵢlⱼ and yₖ=−1 otherwise.
Train the G PSL using this set.
   For a new image I,
For each of the p semantic classes, select r representative images: {I₁ . . . Iᵣ} where I(Iᵢ, yᵢ) is such that yᵢ=p. This generates a set of q=pr images.
Compute a set of pairs by combining each representative image with the new image I:{(I, I₁) . . . (I, I_{q})}
Use the trained GPSL to predict which pairs are similar.
For each semantic class, compute the number of similar pairs.
Assign the new image I to the class with the maximum number of votes.

### Experiments

In one embodiment, each image in a pair may be represented by a set of descriptors. For example, MPEG-7 Homogeneous Texture Descriptors (HTD) (Manjunath, B., Ohm, J., Vasudevan, V., Yamada, A.: Color and texture descriptors. IEEE CSVT 11(6) (2001) 703-715), color weighted histograms (WH) and patch intensities (PI). WHs may be generated by dividing the color space into 11 bins, for example, and populating a feature vector with points weighted by their distance from the image center. PIs may be generated by dividing the image into 16 patches, for example, and populating a vector with the mean intensity in each patch. The number of histogram bins and patches may be determined empirically. A nonsymmetric pair may consist of two sets of these descriptors stacked together. For the symmetric representation, descriptors element-wise squared difference may be carried out between the two sets. A chi-squared base kernel may be used for WH and a polynomial base kernel of order 1 may be used for the other two descriptors.

Experiments validate that MLPK with a non-symmetric representation is better than using a nonsymmetric kernel with a symmetric representation. Further, with three example algorithms for comparison: SVM with a base kernel, SimpleMKL using MLPK generated from the same base kernel (a total of m kernels) and GPSL (a total of 3m kernels also calculated from the same base kernel). A 5-fold CV may be applied to all three algorithms using all combinations of the three descriptors. It was observed that GPSL outperforms SVM with a base kernel in all cases. SimpleMLK with MLPK also performs better than SVM with a base kernel in all cases, except the HTD descriptor.

Experiments were also preformed for classifying mild vs. severe lesions. For example, three types of features were extracted: Haralick texture descriptor and Cross Correlation responses of the blue and green bands with the same bands of a template lesion image. Three classification experiments were compared: SVM with each descriptor separately (SVMSeparate) to directly classify lesion images, SVM with all features combined by SimpleMKL (SVM-MKL) to directly classify lesion images and finally with GPSLVote (which uses pairwise similarity learning). CV in all cases was performed on a “leave-two-out” basis, where the testing set was made up of one image from each class. All other images formed the training set. In the case of GPSL-Vote, the similarity training dataset may be generated using all combinations of pairs which are in the training set. It was observed that the SVM-MKL algorithm does only as well as the best classifier. However, GPSL-vote may outperforms this, even for a small dataset with a small number of features.

### Exemplary Computer System

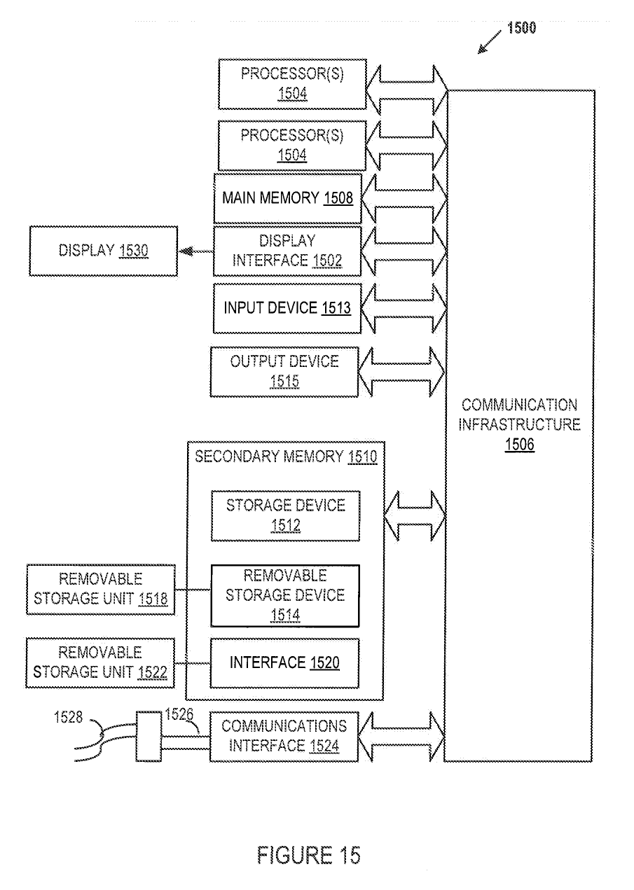

FIG. 15 depicts an illustrative computer system that may be used in implementing an embodiment of the present invention. Specifically, FIG. 15 depicts an embodiment of a computer system 1500 that may be used in computing devices such as, e.g., but not limited to, standalone or client or server devices. FIG. 15 depicts an embodiment of a computer system that may be used as client device, or a server device, etc. The present invention (or any part(s) or function(s) thereof) may be implemented using hardware, software, firmware, or a combination thereof and may be implemented in one or more computer systems or other processing systems. In fact, in one embodiment, the invention may be directed toward one or more computer systems capable of carrying out the functionality described herein. An example of a computer system 1500 is shown in FIG. 15, depicting an embodiment of a block diagram of an illustrative computer system useful for implementing the present invention. Specifically, FIG. 15 illustrates an example computer 1500, which in an embodiment may be, e.g., (but not limited to) a personal computer (PC) system running an operating system such as, e.g., (but not limited to) MICROSOFT® WINDOWS® NT/98/2000/XP/Vista/Windows 7/etc. available from MICROSOFT® Corporation of Redmond, Wash., U.S.A. However, the invention is not limited to these platforms. Instead, the invention may be implemented on any appropriate computer system running any appropriate operating system. In one embodiment, the present invention may be implemented on a computer system operating as discussed herein. An illustrative computer system, computer 1500 is shown in FIG. 15. Other components of the invention, such as, e.g., (but not limited to) a computing device, an imaging device, an imaging system, a communications device, a telephone, a personal digital assistant (PDA), a personal computer (PC), a handheld PC, a laptop computer, a netbook, client workstations, thin clients, thick clients, proxy servers, network communication servers, remote access devices, client computers, server computers, routers, web servers, data, media, audio, video, telephony or streaming technology servers, etc., may also be implemented using a computer such as that shown in FIG. 15.

The computer system 1500 may include one or more processors, such as, e.g., but not limited to, processor(s) 1504. The processor(s) 1504 may be connected to a communication infrastructure 1506 (e.g., but not limited to, a communications bus, cross-over bar, or network, etc.). Processors 1504 may also include multiple independent cores, such as a dual-core processor or a multi-core processor. Processors 1504 may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

Computer system 1500 may include a display interface 1502 that may forward, e.g., but not limited to, graphics, text, and other data, etc., from the communication infrastructure 1506 (or from a frame buffer, etc., not shown) for display on the display unit 1530.

The computer system 1500 may also include, e.g., but is not limited to, a main memory 1508, random access memory (RAM), and a secondary memory 1510, etc. The secondary memory 1510 may include, for example, (but is not limited to) a hard disk drive 1512 and/or a removable storage drive 1514, representing a floppy diskette drive, a magnetic tape drive, an optical disk drive, a compact disk drive CD-ROM, etc. The removable storage drive 1514 may, e.g., but is not limited to, read from and/or write to a removable storage unit 1518 in a well known manner. Removable storage unit 1518, also called a program storage device or a computer program product, may represent, e.g., but is not limited to, a floppy disk, magnetic tape, optical disk, compact disk, etc. which may be read from and written to removable storage drive 1514. As will be appreciated, the removable storage unit 1518 may include a computer usable storage medium having stored therein computer software and/or data.

In alternative embodiments, secondary memory 1510 may include other similar devices for allowing computer programs or other instructions to be loaded into computer system 1500. Such devices may include, for example, a removable storage unit 1522 and an interface 1520. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units 1522 and interfaces 1520, which may allow software and data to be transferred from the removable storage unit 1522 to computer system 1500.

Computer 1500 may also include an input device such as, e.g., (but not limited to) a mouse or other pointing device such as a digitizer, and a keyboard or other data entry device (none of which are labeled). Other input devices 1513 may include a facial scanning device or a video source, such as, e.g., but not limited to, fundus imager, a retinal scanner, a web cam, a video camera, or other camera.

Computer 1500 may also include output devices, such as, e.g., (but not limited to) display 1530, and display interface 1502. Computer 1500 may include input/output (I/O) devices such as, e.g., (but not limited to) communications interface 1524, cable 1528 and communications path 1526, etc. These devices may include, e.g., but are not limited to, a network interface card, and modems (neither are labeled). Communications interface 1524 may allow software and data to be transferred between computer system 1500 and external devices.

In this document, the terms “computer program medium” and “computer readable medium” may be used to generally refer to media such as, e.g., but not limited to removable storage drive 1514, and a hard disk installed in hard disk drive 1512, etc. These computer program products may provide software to computer system 1500. Some embodiments of the invention may be directed to such computer program products. References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may. In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other.

An algorithm is here, and generally, considered to be a self-consistent sequence of acts or operations leading to a desired result. These include physical manipulations of physical quantities. Usually, though not necessarily, these quantities take the form of electrical or magnetic data capable of being stored, transferred, combined, compared, and otherwise manipulated. It has proven convenient at times, principally for reasons of common usage, to refer to these data as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments of the present invention may include apparatuses for performing the operations herein. An apparatus may be specially constructed for the desired purposes, or it may comprise a general purpose device selectively activated or reconfigured by a program stored in the device.

In yet another embodiment, the invention may be implemented using a combination of any of, e.g., but not limited to, hardware, firmware and software, etc.

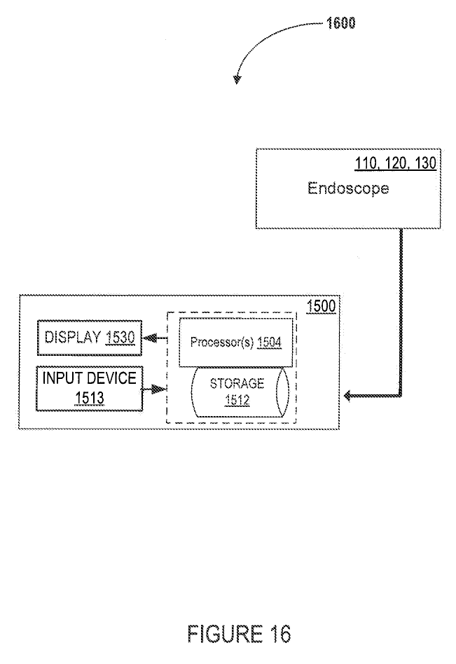

FIG. 16 depicts an illustrative imaging capture and image processing and/or archiving system 1600. 1600 includes an endoscope 110, 120, 130 that is capable of taking endoscopic images and transmitting them to computing system 1500. Different embodiments of the invention include different endoscope devices including a wireless capsule endoscopy device, a flexible endoscope, a contact hysteroscope, a flexible borescope, a video borescope, a rigid borescope, a pipe borescope, a GRIN lens endoscope, or a fibroscope. 1600 also includes a processing unit 1500. 1500 is a computing system such as depicted in FIG. 15. 1500 may be an image processing system and/or image archiving system and is capable of receiving image data as input. 1600 may include a storage device 1512, one or more processors 1504, a display device 1530, and an input device 1513.

In one embodiment of the invention, the processing unit 1500 is capable of processing the received images. Such processing includes detecting an attribute of interest, determining whether an attribute of interest is present in the images based on a predetermined criterion, classifying a set of images that contains at least one attribute of interest, and classifying another set of images that does not contain at least one attribute of interest. The attribute of interest may be a localized region of interest that contains a disease relevant visual attribute. The disease relevant visual attribute include endoscopic images that include images of a lesion, a polyp, bleeding, inflammation, discoloration, and/or stenosis.

The processing unit 1500 may also detect duplicate attribute of interest in multiple endoscopic images. The processing unit 1500 may identify an attribute of interest in a first image that corresponds to an attribute of interest of a second image. Once duplicates are identified, the processing unit 1500 may remove the duplicates from an image set.

The system 1600 displays result data on display 1530. The result data includes the classified images containing an attribute of interest. The system 1600 may allow relevance feedback through an input device 1513. The relevance feedback includes a change to the result data. The system 1600 will use the relevance feedback to train the classifiers. Relevance feedback may include a change in said classification, a removal of the image from said reduced set of images, a change in an ordering of said reduced set of images, an assignment of an assessment attribute, and/or an assignment of a measurement. The system 1600 training may be performed using artificial neural networks, support vector machines, and/or linear discriminant analysis.

The attribute of interest in the images may correspond to some type of abnormality. The system 1600 will perform an assessment of the severity of each said attribute of interest. The assessment includes a score, a rank, a structured assessment comprising of one or more categories, a structured assessment on a Likert scale, and/or a relationship with one or more other images, wherein said relationship comprises less severe or more severe. The system 1600 may derive an overall score for the image set containing at least one attribute of interest based on the severity of each said region of interest. The score may be based on the Lewis score, the Crohn's Disease Endoscopy Index of Severity, the Simple Endoscopic Score for Crohn's Disease, the Crohn's Disease Activity Index, and/or another rubric based on image appearance attributes. The appearance attributes include lesion exudates, inflammation, color, and/or texture.

The system 1600 may also identify images that are unusable and remove those images from further processing. The images may be unusable because they contain extraneous particles in the image. Such extraneous information includes air bubbles, food, fecal matter, normal tissue, non-lesion, and/or structures.

The system 1600 may use supervised machine learning, unsupervised machine learning, or both during the processing of the images. The system 1600 may also use statistical measures, machine learning algorithms, traditional classification techniques, regression techniques, feature vectors, localized descriptors, MPEG-7 visual descriptors, edge features, color histograms, image statistics, gradient statistics, Haralick texture features, dominant color descriptors, edge histogram descriptors, homogeneous texture descriptors, spatial kernel weighting, uniform grid sampling, grid sampling with multiple scales, local mode-seeking using mean shift, generic lesion templates, linear discriminate analysis, logistic regression, K-nearest neighbors, relevance vector machines, expectation maximation, discrete wavelets, and/or Gabor filters. System 1600 may also use measurements of color, texture, hue, saturation, intensity, energy, entropy, maximum probability, contrast, inverse difference moment, and/or correlation. System 1600 may also use meta methods, boosting methods, bagging methods, voting, weighted voting, adaboost, temporal consistency, performing a second classification procedure on data neighboring said localized region of interest, and/or Bayesian analysis.

In one embodiment, the images taken by the endoscope are images taken within a gastrointestinal track and the attribute of interest includes an anatomic abnormality in the gastrointestinal track. The abnormality comprises includes a lesion, mucosal inflammation, an erosion, an ulcer, submucosal inflammation, a stricture, a fistulae, a perforation, an erythema, edema, blood, and/or a boundary organ.

In one embodiment, system 1600 receives and processes images in real-time from the endoscope. This may be the scenario where a surgeon or clinician is manually operating the endoscope. In another embodiment, system 1600 is processing the images that are stored in a database of images. This may be the scenario where a capsule endoscopic device is transmitting images to data storage for later processing.

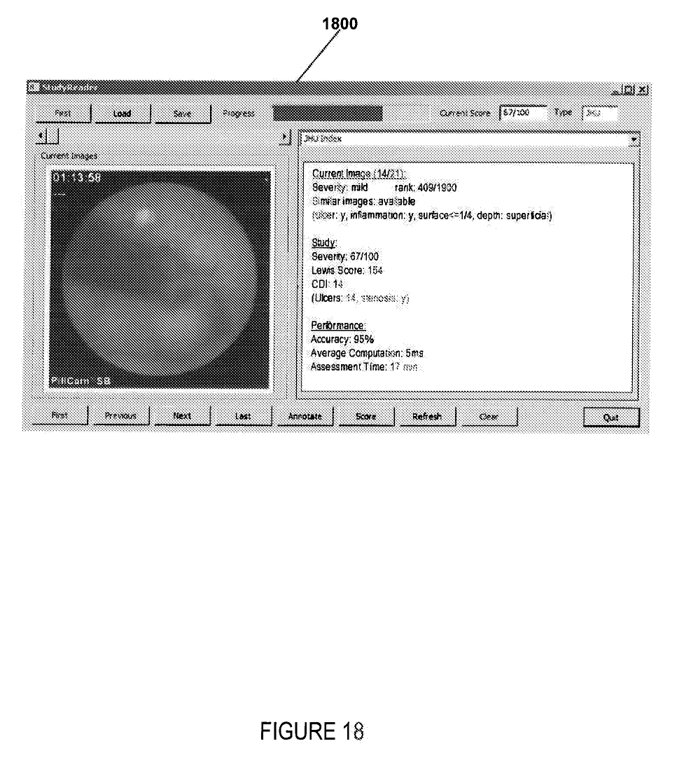

FIG. 18 depicts an illustrative screen shot of a user interface application 1800 designed to support review of imaging data. The software should have, at least, the following features:
Study Review: The ability to review, store, and recall identified or de-identified studies (in randomized and blind fashion). This may be either lesion thumbnails (selected images) and associated data, or an entire CE study as a single image stream.
Clinical Review: The ability to review, edit, and export identified or de-identified clinical data relevant to diagnosis.
Longitudinal Review: The ability to relate studies linked together by the patient ID.
Study Annotation: The ability to annotate, review, and export annotated information, including regions of interest and landmarks.
Study Scoring: The ability to assign scores, using multiple alphanumeric scoring methods including the CDAI and the Lewis score, both individual lesions, and a study as appropriate.
Assessment: The ability to automatically assess, and manually adjust severity of lesions, and studies using detection, classification, and severity rating methods
The current invention is not limited to the specific embodiments of the invention illustrated herein by way of example, but is defined by the claims. One of ordinary skill in the art would recognize that various modifications and alternatives to the examples discussed herein are possible without departing from the scope and general concepts of this invention.

We claim:

1. An automated method of processing images from an endoscope comprising:
receiving a plurality of endoscopic images by an image processing system;
processing each of said plurality of endoscopic images with said image processing system to determine whether at least one attribute of interest is present in each image that satisfies a predetermined criterion; and
classifying said plurality of endoscopic images into a reduced set of images each of which contains at least one attribute of interest and a remainder set of images each of which is free from said attribute.

2. The automated method according to claim 1, where the attribute of interest is a localized region of interest containing a disease relevant visual attribute.

3. The automated method of claim 2, wherein said disease relevant visual attribute comprises an image of: a lesion, a polyp, bleeding, inflammation, discoloration, or stenosis.

4. The automated method according to claim 1, further comprising:
processing said reduced set of images with said image processing system to identify an attribute of interest in a first image of said reduced set of images that corresponds to an attribute of interest of a second image of said reduced set of images.

5. The automated method according to claim 4, further comprising:
classifying said reduced set of images into a non-redundant set of images such that no attribute of interest of any one of said non-redundant set of images corresponds to an attribute of interest of any other one of said non-redundant set of images.

6. The method according to claim 1, further comprising:
displaying result data with said image processing system, wherein said result data comprises an image from said reduced set of images containing at least one attribute of interest.

7. The method according to claim 6, further comprising:
receiving relevance feedback on said image processing system from an observer of said result data, wherein said relevance feedback comprises a change to said result data; and
training said image processing system based on said received relevance feedback.

8. The method according to claim 7, wherein said relevance feedback includes one or more of the following:
a change in said classification,
a removal of the image from said reduced set of images,
a change in an ordering of said reduced set of images,
an assignment of an assessment attribute, and
an assignment of a measurement.

9. The method according to claim 7, wherein said training comprises using at least one of the following:
artificial neural networks,
support vector machines, and
linear discriminant analysis.

10. The method according to claim 1, wherein said attribute of interest corresponds to an abnormality, said method further comprising:
assessing a severity of each said attribute of interest in said reduced set of images containing at least one attribute of interest using said image processing system.

11. The method according to claim 10, where said assessing comprises calculating one of:
a score,
a rank,
a structured assessment comprising of one or more categories,
a structured assessment on a Likert scale, and
a relationship with one or more other images, wherein said relationship comprises less severe or more severe.

12. The method according to claim 10, further comprising:
deriving a score for said reduced set of images containing at least one attribute of interest based on said severity of each said region of interest using said image processing system.

13. The method according to claim 12, wherein said score comprises at least one of:
a Lewis score,
a Crohn's Disease Endoscopy Index of Severity,
a Simple Endoscopic Score for Crohn's Disease,
a Crohn's Disease Activity index, and
a rubric based on image appearance attributes, wherein said appearance attributes comprises one of: lesion exudates, inflammation, color, and texture.

14. The method according to claim 1, further comprising:
prior to the first said processing, processing each of said plurality of endoscopic images with said image processing system to determine whether any of said plurality of endoscopic images is unusable for further processing; and
removing said unusable image from further processing.

15. The method according to claim 14, wherein said unusable image comprises at least one image of:
air bubbles,
food,
fecal matter,
normal tissue,
non-lesion, and
structures.

16. The method according to claim 1, wherein said processing each of said plurality of endoscopic images and classifying said plurality of endoscopic images comprises at least one of: supervised machine learning and unsupervised machine learning.

17. The method according to claim 1, wherein said processing each of said plurality of endoscopic images comprises using at least one of:
statistical measures,
machine learning algorithms,
traditional classification techniques,
regression techniques,
feature vectors,
localized descriptors,
MPEG-7 visual descriptors,
edge features,
color histograms,
image statistics,
gradient statistics,
Haralick texture features,
dominant color descriptors,
edge histogram descriptors,
homogeneous texture descriptors,
spatial kernel weighting,
uniform grid sampling,
grid sampling with multiple scales,
local mode-seeking using mean shift,
generic lesion templates,
linear discriminate analysis,
logistic regression,
K-nearest neighbors,
relevance vector machines,
expectation maximation,
discrete wavelets, and
Gabor filters.

18. The method according to claim 1, wherein said predetermined criterion comprises a measurement of at least one of:
color,
texture,
hue,
saturation,
intensity,
energy,
entropy,
maximum probability,
contrast,
inverse difference moment, and
correlation.

19. The method according to claim 1, wherein said classifying said plurality of endoscopic images comprises using at least one of:
meta methods,
boosting methods,
bagging methods,
voting,
weighted voting,
adaboost,
temporal consistency,
performing a second classification procedure on data neighboring said localized region of interest, and
Bayesian analysis.

20. The method according to claim 1, wherein said endoscope comprises at least one of:
a wireless capsule endoscopy device,
an endoscope,
a flexible endoscope,
a contact hysteroscope,
a flexible borescope,
a video borescope,
a rigid borescope,
a pipe borescope,
a GRIN lens endoscope, and
a fibroscope.

21. The method according to claim 1, wherein,
said plurality of endoscopic images are images taken within a gastrointestinal track; and
said attribute of interest comprises an anatomic abnormality in said gastrointestinal track.

22. The method according to claim 21, wherein said anatomic abnormality comprises at least one of:
a lesion,
mucosal inflammation,
an erosion,
an ulcer,
submucosal inflammation,
a stricture,
a fistulae,
a perforation,
an erythema,
edema,
blood, and
a boundary organ.

23. The method according to claim 1, wherein said receiving a plurality of endoscopic images by an image processing system comprises receiving said plurality of endoscopic images from one of:
a database of images, and
in real-time from said endoscope.

24. An endoscopy system, comprising:
an endoscope;
a processing unit in communication with said endoscope, said processing unit comprising executable instructions for detecting an attribute of interest;
wherein said processing unit performs the following in response to receiving a plurality of endoscopic images from said endoscope based on said executable instructions:
a determination of whether at least one attribute of interest is present in each image that satisfies a predetermined criterion; and
a classification of said plurality of endoscopic images into a reduced set of images each of which contains said at least one attribute of interest and a remainder set of images each of which is free from said at least one attribute of interest.

25. The system of claim 24, where the attribute of interest is a localized region of interest containing a disease relevant visual attribute.

26. The system of claim 25, wherein said disease relevant visual attribute comprises an image of: a lesion, a polyp, bleeding, inflammation, discoloration, or stenosis.

27. The system of claim 24, wherein said processing unit further performs the following in response to receiving a plurality of endoscopic images from said endoscope based on said executable instructions:
an identification of an attribute of interest in a first image of said reduced set of images that corresponds to an attribute of interest of a second image of said reduced set of images.

28. The system of claim 27, wherein said processing unit further performs the following in response to receiving a plurality of endoscopic images from said endoscope based on said executable instructions:
a classification of said reduced set of images into a non-redundant set of images such that no attribute of interest of any one of said non-redundant set of images corresponds to an attribute of interest of any other one of said non-redundant set of images.

29. The system of claim 24, further comprising:
a display device; and
wherein said processing unit further performs the following in response to receiving a plurality of endoscopic images from said endoscope based on said executable instructions:
a display of result data on said display device, wherein said result data comprises an image from said reduced set of images containing at least one attribute of interest.

30. The system of claim 29, further comprising:
an input device; and
wherein said processing unit further performs the following in response to receiving a plurality of endoscopic images from said endoscope based on said executable instructions:
a receipt of relevance feedback, wherein said relevance feedback comprises a change to said result data; and
a training of said processing unit based on said received relevance feedback.

31. The system of claim 30, wherein said relevance feedback includes one or more of the following:
a change in said classification,
a removal of the image from said reduced set of images,
a change in an ordering of said reduced set of images,
an assignment of an assessment attribute, and
an assignment of a measurement.

32. The system of claim 30, wherein said training of said processing unit comprises using at least one of the following:
artificial neural networks,
support vector machines, and
linear discriminant analysis.

33. The system of claim 24, wherein
said attribute of interest corresponds to an abnormality; and
wherein said processing unit further performs the following in response to receiving a plurality of endoscopic images from said endoscope based on said executable instructions:
an assessment of a severity of each said attribute of interest in said reduced set of images containing at least one attribute of interest.

34. The system of claim 33, where said assessment comprises calculating one of:
a score,
a rank,
a structured assessment comprising of one or more categories,
a structured assessment on a Likert scale, and
a relationship with one or more other images, wherein said relationship comprises less severe or more severe.

35. The system of claim 33, wherein said processing unit further performs the following in response to receiving a plurality of endoscopic images from said endoscope based on said executable instructions:
a derivation of a score for said reduced set of images containing at least one attribute of interest based on said severity of each said region of interest.

36. The system of claim 35, wherein said score comprises at least one of:
a Lewis score,
a Crohn's Disease Endoscopy Index of Severity,
a Simple Endoscopic Score for Crohn's Disease,
a Crohn's Disease Activity Index, and
a rubric based on image appearance attributes, wherein said appearance attributes comprises one of: lesion exudates, inflammation, color, and texture.

37. The system of claim 24, wherein said processing unit further performs the following in response to receiving a plurality of endoscopic images from said endoscope based on said executable instructions:
an identification of each of said plurality of endoscopic images to determine whether any of said plurality of endoscopic images is unusable for further processing; and
a removal of said unusable image from further processing.

38. The system according to claim 37, wherein said unusable image comprises at least one image of:
air bubbles,
food,
fecal matter,
normal tissue,
non-lesion, and
structures.

39. The system of claim 24, wherein said determination of whether at least one attribute of interest is present and said classification of said plurality of endoscopic images comprises using at least one of: supervised machine learning and unsupervised machine learning.

40. The system of claim 24, wherein said determination of whether at least one attribute of interest is present comprises using at least one of:
statistical measures,
machine learning algorithms,
traditional classification techniques,
regression techniques,
feature vectors,
localized descriptors,
MPEG-7 visual descriptors,
edge features,
color histograms,
image statistics,
gradient statistics,
Haralick texture features,
dominant color descriptors,
edge histogram descriptors,
homogeneous texture descriptors,
spatial kernel weighting,
uniform grid sampling,
grid sampling with multiple scales,
local mode-seeking using mean shift,
generic lesion templates,
linear discriminate analysis,
logistic regression,
K-nearest neighbors,
relevance vector machines,
expectation maximation,
discrete wavelets, and
Gabor filters.

41. The system of claim 24, wherein said predetermined criterion comprises a measurement of at least one of:
color,
texture,
hue,
saturation,
intensity,
energy,
entropy,
maximum probability,
contrast,
inverse difference moment, and
correlation.

42. The system according to claim 24, wherein said classification of said plurality of endoscopic images comprises using at least one of:
meta methods,
boosting methods,
bagging methods,
voting,
weighted voting,
adaboost,
temporal consistency,
performing a second classification procedure on data neighboring said localized region of interest, and
Bayesian analysis.

43. The system of claim 24, wherein said endoscope comprises one of:
a wireless capsule endoscopy device,
a flexible endoscope,
a contact hysteroscope,
a flexible borescope,
a video borescope,
a rigid borescope,
a pipe borescope,
a GRIN lens endoscope, and
a fibroscope.

44. The method according to claim 24, wherein,
said plurality of endoscopic images are images taken within a gastrointestinal track; and
said attribute of interest comprises an anatomic abnormality in said gastrointestinal track.

45. The method according to claim 44, wherein said anatomic abnormality comprises at least one of:
a lesion,
mucosal inflammation,
an erosion,
an ulcer,
submucosal inflammation,
a stricture,
a fistulae,
a perforation,
an erythema,
edema,
blood, and
a boundary organ.

46. The method according to claim 24, wherein said receiving a plurality of images from said endoscope comprises receiving images from one of:
a database of endoscopic images, and
in real-time from said endoscope.

47. A computer readable medium storing executable instructions for execution by a computer having memory, the medium storing instructions for:
receiving a plurality of endoscopic images;
processing each of said plurality of endoscopic images to determine whether at least one attribute of interest is present in each image that satisfies a predetermined criterion; and
classifying said plurality of endoscopic images into a reduced set of images each of which contains said at least one attribute of interest and a remainder set of images each of which is free from said at least one attribute of interest.
